(19) United States
(12) Patent Application Publication
(10) Pub. No.: US 2023/0002831 A1
(43) Pub. Date: Jan. 5, 2023

(54) METHODS AND COMPOSITIONS FOR ANALYSES OF CANCER

(71) Applicant: The Johns Hopkins University, Baltimore, MD (US)

(72) Inventors: Victor Velculescu, Dayton, MD (US); Alessandro Leal, Baltimore, MD (US); Jillian Phallen, Baltimore, MD (US); Valsamo Anagnostou, Baltimore, MD (US); Remond Fijneman, Amsterdam (NL); Gernt Meijer, Amsterdam (NL); Nicole Van Grieken, Amsterdam (NL)

(21) Appl. No.: 17/779,936

(22) PCT Filed: Nov. 25, 2020
(86) PCT No.: PCT/US2020/062312

§ 371 Date: May 25, 2022

Related U.S. Application Data
(60) Provisional application No. 62/940,210, filed on Nov. 25, 2019.

(51) Int. Cl. C12Q 1/6886; G16B 20/20

(52) U.S. Cl. CPC C12Q 1/6886; G16B 20/20; C12Q 2600/156

(57) ABSTRACT

Combined ultrasensitive sequencing of matched white blood cells and cell free DNA (cfDNA) identified bona fide tumor-specific alterations that predict clinical outcome after preoperative treatment and resection.

### CROSS-REFERENCE TO RELATED APPLICATION

This application claims the benefit of U.S. provisional application No. 62/940,210 filed Nov. 25, 2019, which is incorporated by reference herein in its entirety.

### STATEMENT OF GOVERNMENT RIGHTS

This invention was made with government support under grant CA121113 and CA180950 awarded by the National Institutes of Health. The government has certain rights in the invention.

### FIELD OF THE INVENTION

Embodiments of the invention are directed to non-invasive methods for detecting and identifying tumor-specific alterations in the circulation of a subject. In part, the methods provide a longitudinally assessment of a patient's response to therapy, recurrence and/or survivability.

### BACKGROUND

A major challenge after multimodal curative treatment for resectable gastric cancer is identifying patients with microscopic residual disease at high risk of recurrence after surgery (Marrelli, D. et al. Ann Surg 241, 247-255, doi:10.1097/01.sla.0000152019.14741.97 (2005). Songun, I., et al. Lancet Oncol 11, 439-449, doi:10.1016/S1470-2045(10)70070-X (2010). Bickenbach, K. A., et al. Ann Surg Oncol 20, 2663-2668, doi:10.1245/s10434-013-2950-5 (2013). Van Cutsem, E., et al. Lancet 388, 2654-2664, doi:10.1016/S0140-6736(16)30354-3 (2016)). Currently available imaging techniques and traditional blood biomarkers to capture minimal residual disease (MRD) state after surgery have poor sensitivity and do not play a role in clinical practice (Aurello, P. et al. World journal of gastroenterology 23, 3379-3387, doi:10.3748/wjg.v23.i19.3379 (2017)). Histopathological assessment of the effects of neoadjuvant chemotherapy on resection specimens has become an important tool to provide prognostic information (Becker, K. et al. Cancer 98, 1521-1530, doi:10.1002/cncr.11660 (2003). Smyth, E. C. et al. J Clin Oncol 34, 2721-2727, doi:10.1200/JCO.2015.65.7692 (2016). Langer, R. & Becker, K. Tumor regression grading of gastrointestinal cancers after neoadjuvant therapy. Virchows Archiv: an international journal of pathology 472, 175-186, doi:10.1007/s00428-017-2232-x (2018)). However, microscopic residual tumor, lymph node infiltration, and poor histopathological response do not measure the real-time presence of residual disease.

### SUMMARY

We now provide new non-invasive methods for detecting and identifying tumor-specific alterations in the circulation of a subject. In one aspect, the methods include matched white-blood cell and cell-free DNA analyses for detection of mutations in circulating tumor DNA of patients with cancer. Tumor-specific alterations can be detected and monitored over different time points, in response to treatments and the like.

Methods and systems of the invention are particularly useful for detecting and monitoring patients suffering from or susceptible to gastric cancer. Methods and systems of the invention are also particularly useful for detecting and monitoring patients suffering from or susceptible to colorectal cancer. Methods and systems of the invention are also particularly useful for detecting and monitoring patients suffering from or susceptible to lung cancer. Methods and systems of the invention are also particularly useful for detecting and monitoring patients suffering from or susceptible to an esophageal cancer.

Accordingly, in certain embodiments, a method is provided of detecting tumor specific mutations in a subject's circulating tumor DNA, the method comprising obtaining whole blood from a subject, separating the plasma and cellular components and extracting the DNA from each; preparing sequencing libraries of genomic DNA comprising cell free DNA (cfDNA) and cellular DNA obtained from a sample of the subject's whole blood; identifying sequence variations in the cfDNA and cellular DNA as compared to a reference genomic sequence; comparing the sequence variations of cfDNA and cellular DNA; thereby, identifying tumor specific mutations. In some embodiments, the sequence reads are generated from a next generation sequencing (NGS) procedure.

In certain embodiments, a method based on matched white-blood cell and cell-free DNA analyses for detection of mutations in circulating tumor DNA of patients with cancer, can be determinative of eligibility for systemic therapy with anti-cancer agents.

In certain embodiments, the method provides for detection of mutations in circulating tumor DNA of patients with cancer eligible for surgical resection.

In certain embodiments, the method provides for detection of changes in levels of circulating tumor DNA in patients treated with perioperative chemotherapy.

In certain embodiments, the method provides for detection of changes in levels of circulating tumor DNA in patients treated with neoadjuvant anti-cancer agents.

In certain embodiments, preferred methods provide for prediction of pathological response to preoperative chemotherapy in patients with cancer. Such methods are particularly useful for patients with gastric cancer. Such methods also are particularly useful for patients with colorectal cancer, lung cancer, and/or esophageal cancer.

In certain embodiments, preferred method provide for prediction of recurrence after perioperative treatment in patients with cancer.

In certain embodiments, preferred method provide for prediction of cancer-specific survival after perioperative treatment in patients with cancer.

In certain embodiments, preferred method provide for prediction of overall survival after perioperative treatment in patients with cancer.

In certain embodiments, preferred methods provide for detection of minimal residual disease after tumor resection in patients with cancer.

In certain embodiments, preferred methods provide for detection of alterations associated with clonal hematopoiesis in patients with cancer, eligible for systemic treatment with anti-cancer agents.

In certain embodiments, preferred methods provide for the identification of patients that will benefit from receiving neoadjuvant systemic treatment before tumor resection.

In certain embodiments, the tumor type is gastric cancer. In additional embodiments, the tumor type is colorectal cancer. In other embodiments, the tumor type is lung cancer. In other embodiments, the tumor type is esophageal cancer.

In certain embodiments, the circulating tumor DNA is analyzed before therapy, at the time of surgery, and within two months after surgery.

### Definitions

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value or range. Alternatively, particularly with respect to biological systems or processes, the term can mean within an order of magnitude within 5-fold, and also within 2-fold, of a value. Where particular values are described in the application and claims, unless otherwise stated the term “about” meaning within an acceptable error range for the particular value should be assumed.

The terms “aligned”, “alignment”, “mapped” or “aligning”, “mapping” refer to one or more sequences that are identified as a match in terms of the order of their nucleic acid molecules to a known sequence from a reference genome. Such alignment can be done manually or by a computer algorithm, examples including the Efficient Local Alignment of Nucleotide Data (ELAND) computer program distributed as part of the Illumina Genomics Analysts pipeline. The matching of a sequence read in aligning can be a 100% sequence match or less than 100% (non-perfect match).

The term “alternative allele” or “ALT” refers to an allele having one or more mutations relative to a reference allele, e.g., corresponding to a known gene.

The term “cancer” as used herein is meant, a disease, condition, trait, genotype or phenotype characterized by unregulated cell growth or replication as is known in the art; including gastric cancer, colorectal cancer, lung cancer, colorectal cancer, lung cancer, esophageal cancer.as well as, for example, leukemias, e.g., acute myelogenous leukemia (AML), chronic myelogenous leukemia (CML), acute lymphocytic leukemia (ALL), and chronic lymphocytic leukemia, AIDS related cancers such as Kaposi's sarcoma; breast cancers; bone cancers such as Osteosarcoma, Chondrosarcomas, Ewing's sarcoma, Fibrosarcomas, Giant cell tumors, Adamantinomas, and Chordomas; Brain cancers such as Meningiomas, Glioblastomas, Lower-Grade Astrocytomas, Oligodendrocytomas, Pituitary Tumors, Schwannomas, and Metastatic brain cancers; cancers of the head and neck including various lymphomas such as mantle cell lymphoma, non-Hodgkins lymphoma, adenoma, squamous cell carcinoma, laryngeal carcinoma, gallbladder and bile duct cancers, cancers of the retina such as retinoblastoma, cancers of the esophagus, gastric cancers, multiple myeloma, ovarian cancer, uterine cancer, thyroid cancer, testicular cancer, endometrial cancer, melanoma, lung cancer, bladder cancer, prostate cancer, lung cancer (including non-small cell lung carcinoma), pancreatic cancer, sarcomas, Wilms' tumor, cervical cancer, head and neck cancer, skin cancers, nasopharyngeal carcinoma, liposarcoma, epithelial carcinoma, renal cell carcinoma, gallbladder adeno carcinoma, parotid adenocarcinoma, endometrial sarcoma, multidrug resistant cancers; and proliferative diseases and conditions, such as neovascularization associated with tumor angiogenesis.

The term “candidate variant,” “called variant,” or “putative variant” refers to one or more detected nucleotide variants of a nucleotide sequence, for example, at a position in the genome that is determined to be mutated. Generally, a nucleotide base is deemed a called variant based on the presence of an alternative allele on sequence reads obtained from a sample, where the sequence reads each cross over the position in the genome. The source of a candidate variant may initially be unknown or uncertain. During processing, candidate variants may be associated with an expected source such as genomic DNA (e.g., blood-derived) or cells impacted by cancer (e.g., tumor-derived). Additionally, candidate variants may be called as true positives. A variant of interest is particular variant of a genetic sequence that is to be measured, qualified, quantified, or detected. In some implementations, a variant of interest is a variant known or suspected to be associated with a condition, such as a cancer, a tumor, or a genetic disorder.

The term “cell free nucleic acid,” “cell free DNA,” or “cfDNA” refers to nucleic acid fragments that circulate in an individual's body (e.g., bloodstream) and originate from one or more healthy cells and/or from one or more cancer cells. Additionally cfDNA may come from other sources such as viruses, fetuses, etc.

The term “circulating tumor DNA” or “ctDNA” refers to nucleic acid fragments that originate from tumor cells or other types of cancer cells, which may be released into an individual's bloodstream as result of biological processes such as apoptosis or necrosis of dying cells or actively released by viable tumor cells.

As used herein, the terms “comprising,” “comprise” or “comprised,” and variations thereof, in reference to defined or described elements of an item, composition, apparatus, method, process, system, etc. are meant to be inclusive or open ended, permitting additional elements, thereby indicating that the defined or described item, composition, apparatus, method, process, system, etc. includes those specified elements— or, as appropriate, equivalents thereof— and that other elements can be included and still fall within the scope/definition of the defined item, composition, apparatus, method, process, system, etc.

“Diagnostic” or “diagnosed” means identifying the presence or nature of a pathologic condition. Diagnostic methods differ in their sensitivity and specificity. The “sensitivity” of a diagnostic assay is the percentage of diseased individuals who test positive (percent of “true positives”). Diseased individuals not detected by the assay are “false negatives.” Subjects who are not diseased and who test negative in the assay, are termed “true negatives.” The “specificity” of a diagnostic assay is 1 minus the false positive rate, where the “false positive” rate is defined as the proportion of those without the disease who test positive. While a particular diagnostic method may not provide a definitive diagnosis of a condition, it suffices if the method provides a positive indication that aids in diagnosis.

An “effective amount” as used herein, means an amount which provides a therapeutic or prophylactic benefit.

The term “genomic nucleic acid,” or “genomic DNA,” refers to nucleic acid including chromosomal DNA that originates from one or more healthy (e.g., non-tumor) cells. In various embodiments, genomic DNA can be extracted from a cell derived from a blood cell lineage, such as a white blood cell (WBC).

The term “Next Generation Sequencing (NGS)” herein refers to sequencing methods that allow for massively parallel sequencing of clonally amplified molecules and of single nucleic acid molecules. See, for example, Phallen, J. et al. Direct detection of early-stage cancers using circulating tumor DNA. Sci Transl Med 9, doi:10.1126/scitranslmed.aan2415 (2017). Li B. T. et al. Annals of Oncology 30: 597-603, 2019, doi:10.1093/annonc/mdz04, incorporated by reference in their entirety. Non-limiting examples of NGS include sequencing-by-synthesis using reversible dye terminators, and sequencing-by-ligation.

“Optional” or “optionally” means that the subsequently described event or circumstance can or cannot occur, and that the description includes instances where the event or circumstance occurs and instances where it does not.

As used in this specification and the appended claims, the term “or” is generally employed in its sense including “and/or” unless the content clearly dictates otherwise.

“Parenteral” administration of an immunogenic composition includes, e.g., subcutaneous (s.c.), intravenous (i.v.), intramuscular (i.m.), or intrasternal injection, or infusion techniques.

The terms “patient” or “individual” or “subject” are used interchangeably herein, and refers to a mammalian subject to be treated, with human patients being preferred. In some cases, the methods of the invention find use in experimental animals, in veterinary application, and in the development of animal models for disease, including, but not limited to, rodents including mice, rats, and hamsters, and primates.

The term “reference genome” as used herein may refer to a digital or previously identified nucleic acid sequence database, assembled as a representative example of a species or subject. Reference genomes may be assembled from the nucleic acid sequences from multiple subjects, sample or organisms and does not necessarily represent the nucleic acid makeup of a single person. Reference genomes may be used to for mapping of sequencing reads from a sample to chromosomal positions. For example, a reference genome used for human subjects as well as many other organisms is found at the National Center for Biotechnology Information at ncbi.nlm.nih.gov.

The term “read segment” or “read” refers to any nucleotide sequences including sequence reads obtained from an individual and/or nucleotide sequences derived from the initial sequence read from a sample obtained from an individual.

The term “sequence reads” refers to nucleotide sequences read from a sample obtained from an individual. Sequence reads can be obtained through various methods known in the art.

As defined herein, a “therapeutically effective” amount of a compound or agent (i.e., an effective dosage) means an amount sufficient to produce a therapeutically (e.g., clinically) desirable result. The compositions can be administered from one or more times per day to one or more times per week; including once every other day. The skilled artisan will appreciate that certain factors can influence the dosage and timing required to effectively treat a subject, including but not limited to the severity of the disease or disorder, previous treatments, the general health and/or age of the subject, and other diseases present. Moreover, treatment of a subject with a therapeutically effective amount of the compounds of the invention can include a single treatment or a series of treatments.

As used herein, the terms “treat,” “treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

Genes: All genes, gene names, and gene products disclosed herein are intended to correspond to homologs from any species for which the compositions and methods disclosed herein are applicable. It is understood that when a gene or gene product from a particular species is disclosed, this disclosure is intended to be exemplary only, and is not to be interpreted as a limitation unless the context in which it appears clearly indicates. Thus, for example, for the genes or gene products disclosed herein, are intended to encompass homologous and/or orthologous genes and gene products from other species.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIGS. 1A and 1B show the analysis of cfDNA in patients with resectable gastric cancer. FIG. 1A: Study schematic. Patients with confirmed stage IB-IVA gastric adenocarcinoma eligible for perioperative treatment with systemic chemotherapy were randomized upfront to receive three cycles of preoperative chemotherapy followed by three cycles of postoperative chemotherapy or to receive the same preoperative regimen followed by postoperative radiotherapy combined with chemotherapy. A blood draw was collected for each patient at the time of study enrollment (baseline), after three cycles of preoperative chemotherapy (preoperative timepoint), and after surgery (postoperative timepoint). Blood samples were initially processed to allow proper extraction of cfDNA from plasma and genomic DNA (gDNA) from white blood cells (WBC). Both cfDNA and WBC gDNA libraries were hybrid captured with custom RNA oligo pools encompassing 80,930 bases across 58 cancer driver genes. Capture libraries were sequenced at high coverage (>30,000×), followed by sequence alignment, error correction, and variant calling. Mutations detected in WBCs were identified in cfDNA and removed, allowing the identification of tumor-specific mutations in cfDNA. FIG. 1B: Clinicopathological characteristics, type of surgical treatment, pathological stage, and type of postoperative treatment for the subset of patients analyzed in this translational study (n=50). MSI, microsatellite instability; MSS, microsatellite stability; EBV, Epstein-Barr virus; ECC, epirubicin, cisplatin, capecitabine; CC, cisplatin, capecitabine.

FIGS. 2A-2H are a series of plots and schematics demonstrating the identification of white blood cell and ctDNA variants in cfDNA of patients with localized gastric cancer. FIGS. 2A-2B: Ultrasensitive targeted sequencing were used to detect mutations in cfDNA (FIG. 2A) and WBCs (FIG. 2B) in 50 patients, with only those cases having alterations indicated. FIG. 2C: Tumor-specific mutations in ctDNA were identified in 27 individuals after subtraction of WBC derived variants in cfDNA. FIG. 2D: Density plots showing the mutant allele fraction distribution of cfDNA variants (top, yellow), WBC variants (middle, purple), and resulting ctDNA variants (bottom, blue). FIG. 2E: Levels of mutant allele fractions in WBCs (horizontal axis) and their correspondent levels in cfDNA (vertical axis) suggest that WBC alterations are identified at similar levels in cfDNA (Pearson correlation coefficient=0.91, p<0.001). The probability that an identified variant is tumor-derived when the alteration is not detected in WBCs is indicated by the shading of the blue dots. FIG. 2F: Association between age (horizontal axis) and absolute number of WBC variants detected in each patient (vertical axis) suggests that the number of WBC alterations increase with age (r2=0.36, exponential correlation). FIG. 2G, Positions and frequencies of mutations in TP53 detected in cfDNA (top plot) and WBCs (bottom plot) demonstrate that the majority of TP53 alterations in cfDNA are from WBCs. One TP53 splice site mutation detected in both datasets is not shown. FIG. 2H: Cumulative fraction of cfDNA fragments based on cfDNA fragment length (bp) shows an altered distribution for cfDNA fragments harboring tumor-derived TP53 alterations (blue) compared to WBC TP53 variants (red) and wild-type TP53 sequences (p<0.001, Kolmogorov-Smirnov test).

FIGS. 3A-3I are a series of plots, graphs, schematics and H&E images demonstrating preoperative ctDNA as a biomarker for pathologic response and clinical outcome in gastric cancer. FIGS. 3A-3B: Levels of ctDNA variants at baseline and the preoperative timepoint in a molecular responder (CGST33) (FIG. 3A) and in a non-responder (CGST110) (FIG. 3B). Variant MAFs in the molecular responder show elimination of ctDNA, while ctDNA levels are relatively unchanged in the molecular non-responder. A representative H&E image (20× magnification) depicting Mandard's tumor regression grade is shown for each case on the right. FIG. 3C: Heatmap representing the pathological features (TRG and lymph node status) and highest mutant allele fraction detected for each of the 43 patients that underwent surgical resection. Lauren's classifications are depicted in the bottom for each case. FIG. 3D: Dichotomized association between degree of tumor regression and ctDNA status at the preoperative timepoint (p=0.03, Fisher's exact test), FIG. 3E, between degree of tumor regression and disease recurrence (p=0.03, Fisher's exact test), FIG. 3F, between pathological lymph node status and disease recurrence (p=0.002, Fisher's exact test), and FIG. 3G, between ctDNA status at the preoperative timepoint and disease recurrence (p=0.02, Fisher's exact test). H-I, Kaplan-Meier estimates for event-free survival of patients with detected versus non-detected variants at the preoperative timepoint using all cfDNA sequence changes (Log-rankp=0.76; HR=0.9; 95% CI=0.4-2.1) (FIG. 3H) or using only ctDNA alterations identified from the WBC-filtered approach (Log-rankp=0.01; HR=3.0; 95% CI=1.3-6.9) (FIG. 3I).

FIGS. 4A-4E are a series of plots, graphs, schematics and H&E images demonstrating assessment of ctDNA as a minimal residual disease biomarker in resectable gastric cancer. FIGS. 4A-4B: Levels of ctDNA variants from baseline to postoperative timepoint in a molecular responder (CGST32) (FIG. 4A) and in a non-responder (CGST68) (FIG. 4B). Variant MAFs in the molecular responder show elimination of ctDNA, while ctDNA levels continue to rise in the molecular non-responder. A representative H&E image (20× magnification) depicting Mandard's tumor regression grade is shown for each case on the right. FIG. 4C: Longitudinal representation of ctDNA results from 20 patients with a postoperative timepoint available. Black vertical line represents the time of surgery. Green arrows depict patients with no evidence of disease at last follow-up. FIG. 4D-4E, Kaplan-Meier estimates for overall survival of patients with detected versus non-detected variants at the postoperative timepoint using all cf-DNA sequence changes (Log-rankp=0.28; HR=3.3; 95% CI=0.4-29) (FIG. 4D) or using only ctDNA alterations identified from the WBC-filtered approach (Log-rankp=0.001; HR=21.8; 95% CI=3.9-123.1) (FIG. 4E).

FIG. 5 is a schematic representation showing the consort diagram of patients enrolled in the CRITICS trial cohort. Patients with operable stage IB-IVA gastric cancer (n=788) were initially treated with three cycles of preoperative chemotherapy with epirubicin, cisplatin or oxaliplatin, and oral capecitabine followed by surgery. They were randomly assigned to receive postoperative treatment with the same chemotherapy regimen (n=393) or postoperative radiation with cisplatin and oral capecitabine (n=395). Plasma samples used for ctDNA analyses were provided by study centers in The Netherlands.

FIG. 6 is a schematic showing the theoretical sensitivity of detection of ultra-sensitive NGS approach in gastric cancer. Analyses of TCGA Pan-Cancer Atlas gastric adenocarcinoma cohort showed a theoretical sensitivity of 88% for the 58-gene panel (81 Kb) used in this study, with 385 out of 436 cases potentially identified using this approach.

FIGS. 7A-7F are a series of graphs demonstrating the pathological features and ctDNA levels of gastric cancers analyzed. FIG. 7A: Mutant allele fractions of ctDNA at baseline in patients with diffuse and intestinal subtypes (Wilcoxon rank sum test, p=0.02). FIG. 7B: Mutant allele fractions of ctDNA at baseline in patients with well, moderately, and poorly differentiated tumors (Kruskal-Wallis test, p=0.07). C-D, Kaplan-Meier estimates for event-free survival [Log-rank p=0.90; HR=1.0 (95% CI=0.4-2.1)] (FIG. 7C) and overall survival [Log-rank p=0.59; HR=0.8 [(95% CI=0.3-1.9)] (FIG. 7D) of patients with intestinal and diffuse subtypes. FIGS. 7E-7F: Kaplan-Meier estimates for event-free survival [Log-rank p=0.48; HR=1.3 (95% CI=0.6-3.0)] (FIG. 7E) and overall survival [Log-rank p=0.83; HR=1.1 (95% CI=0.5-2.5)](FIG. 7F) of patients treated with adjuvant chemotherapy or chemo-radiotherapy.

FIGS. 8A-8K are a series of graphs demonstrating the dynamics of white blood cell (WBC) sequence alterations in patients with gastric cancer. Mutant allele fractions of WBC variants were identified in cfDNA in patients without tumor-specific mutations.

FIGS. 9A-9D are a series of graphs demonstrating the survival outcomes based on WBC variants or tumor-specific alterations in cfDNA at the baseline timepoint. FIGS. 9A-9B: Kaplan-Meier estimates for event-free survival [Log-rank p=0.18; HR=1.7 (95% CI=0.8-3.8)] (FIG. 9A) and overall survival [Log-rank p=0.46; HR=1.4 (95% CI=0.6-3.2)] (FIG. 9B) of patients with or without WBCs variants in cfDNA at the time of study enrollment. FIGS. 9C-9D: Kaplan-Meier estimates for event-free survival [Log-rank p=0.45; HR=1.4 (95% CI=0.6-3.0)] (FIG. 9C) and overall survival [Log-rank p=0.55; HR=1.3 (95% CI=0.6-3.0)] (D) of patients with or without ctDNA detected at the time of study enrollment.

FIGS. 10A-10Q are graphs demonstrating the dynamic changes in ctDNA during the preoperative chemotherapy interval. ctDNA alterations in each patient were detected during the preoperative chemotherapy interval after removing WBC variants observed in cf-DNA.

FIGS. 11A-11K are a series of graphs demonstrating the dynamic changes in ctDNA before and after surgery. ctDNA alterations observed for each patient were detected from baseline through postoperative timepoints after removing WBC variants observed in cf-DNA.

FIGS. 12A-12C are a series of plots and a heatmap Pathological response at the preoperative timepoint and survival outcomes. FIG. 12A: Heatmap showing the Spearman's rank correlation coefficients between mutant allele fractions at the preoperative timepoint and pathological features after surgery (ypN, pathological lymph node assessment; ypT, pathological tumor assessment; TRG, tumor regression grade). FIG. 12B: Kaplan-Meier estimates for event-free survival of patients with minor or no pathological response (TRG 3-5) and major pathological responses (TRG 1-2) to preoperative chemotherapy [Log-rankp=0.03; HR=3.6 (95% CI=1.1-11.3)]. FIG. 12C: Kaplan-Meier estimates for event-free survival of patients with (ypN1-3) and without (ypN0) lymph node tumor infiltration [Log-rankp=0.002; HR=4.5 (95% CI=1.8-11.6)].

FIGS. 13A and 13B are plots demonstrating the detection of cfDNA and ctDNA variants at the preoperative timepoint and overall survival. FIGS. 13A-13B, Kaplan-Meier estimates for overall survival of patients with detected versus non-detected variants at the preoperative timepoint using all cfDNA sequence changes [Log-rank p=0.74; HR=0.9 (95% CI=0.4-2.1)](FIG. 13A) or using only ctDNA alterations identified from the WBC-filtered approach [Log-rank p=0.03; HR=2.7 (95% CI=1.1-6.7)] (FIG. 13B).

FIGS. 14A-14B are plots demonstrating the detection of cfDNA and ctDNA variants at the postoperative timepoint and event-free survival. FIG. 14A-14B: Kaplan-Meier estimates for event-free survival of patients with detected versus non-detected variants at the postoperative timepoint using all cfDNA sequence data [Log-rank p=0.28; HR=3.3 (95% CI=0.4-29.3)](FIG. 14A) or using only ctDNA alterations identified from the WBC-filtered approach [Log-rank p<0.001; HR=21.8 (95% CI=3.9-123.1)] (FIG. 14B).

FIG. 15 shows correlation between white blood cell and plasma mutant allele fractions. In the plot, white blood cell mutant allele fractions are plotted on the x axis and plasma mutant allele fractions are plotted on the y axis for baseline, blue, and post resection, orange, timepoints.

FIG. 16. Detection of mutations in baseline and post resection plasma. Mutant allele fractions for alterations detected in the plasma are shown for baseline, pre-resection blood draws (Left) and post-resection blood draws (Right). Mutations are colored based on how they were detected in the blood. The central spine depicts patient stage and whether the patient experienced a clinical recurrence. Mutations above 7% at baseline and 5% post-resection are depicted at those values.

FIGS. 17A-17C. (A) shows biospecimen and clinical metadata collection protocols that allow for collection of serial blood and tissue samples from lung cancer patients treated with immunotherapy. Plasma and matched leukocyte DNA samples were deeply sequenced, clonal hematopoiesis variants were filtered out and ctDNA molecular responses were interpreted with respect to the clinical phenotypes of the patients. (B-C) cfDNA mutations from white blood cells in genes were found not canonically associated with clonal hematopoiesis, showing the importance of deep sequencing of matched leukocyte DNA in order to determine whether cfDNA mutations are tumor or CH-derived.

FIGS. 18A-18D. (A) Liquid biopsy analyses reveal that approximately 50% of cf-DNA alterations are non-tumor derived (germline or CH in origin). (B-C) Appropriate classification of plasma mutations as CH- and tumor-derived allowed for assessment of molecular response patterns and distinguish responders from non-responders. (D) Once CH-derived variants were filtered out, ctDNA molecular responses were reflective of progression (PFS).

FIG. 19. Inclusion of CH-derived and germline mutations did not allow for accurate determination of ctDNA molecular response and ctDNA dynamics were not associated with progression-free and overall survival (top panel). In contrast, when matched leukocyte DNA deep sequencing was employed, variants were accurately classified in tumor-derived and CH-derived categories, which in turn allowed for distinction of molecular ctDNA responders from non-responders. Post filtering, ctDNA responders had a significantly longer progression-free and overall survival (bottom panel).

FIG. 20 shows a plot of where ctDNA dynamics captured by the tumor-derived TP53 G245S mutation were concordant with tumor regression noted at the time of resection (10% residual tumor) only when germline-derived and a TP53 CH-derived variant were excluded by matched leukocyte DNA sequencing analyses.

### DETAILED DESCRIPTION

In one aspect, we now provide a matched cfDNA and white blood cell (WBC) sequencing approach that can accurately detect cell-free DNA (cfDNA) alterations after preoperative chemotherapy and after surgery in patients with resectable gastric cancer. The methods embodied herein, provide that ctDNA detection after completion of preoperative treatment as well as minimal residual disease detection after surgery can predict recurrence and survival in patients with resectable gastric cancer treated with multimodal therapeutic regimens. Preferred methods were able to distinguish ctDNA alterations from cfDNA variants related to clonal hematopoiesis and whether ctDNA elimination before or after surgery can serve as a predictive biomarker of patient outcome to perioperative treatment.

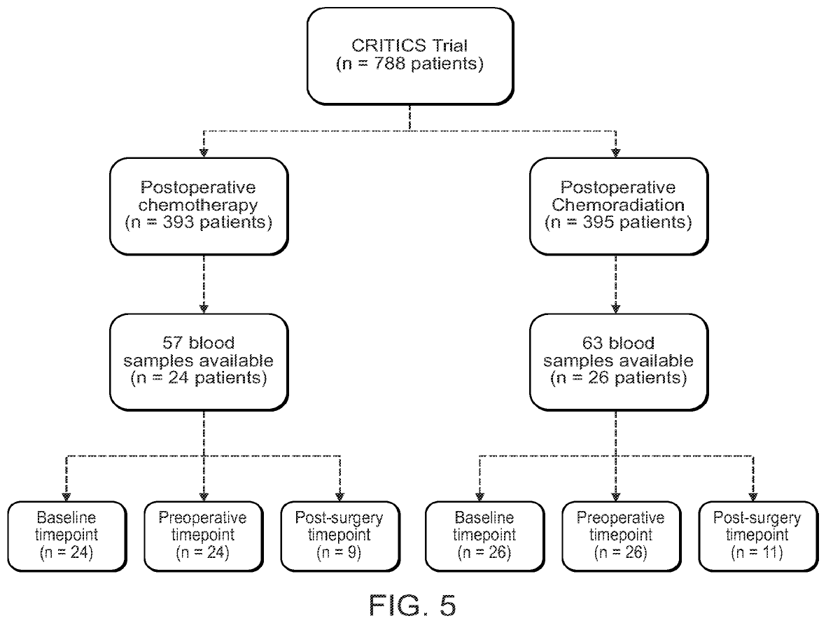

Accordingly, in certain embodiments, methods to identify circulating tumor-derived DNA (ctDNA) alterations include ultrasensitive targeted sequencing analyses of matched cf-DNA and white blood cells from the same patient. The results obtained are described in detail in the examples section which follows. Briefly, samples from patients in the CRITICS trial, a phase III study evaluating perioperative treatment in 788 patients with resectable gastric cancer were analyzed (FIG. 5). Liquid biopsy analyses of 50 patients with available blood samples at multiple time points (n=120) revealed that 52% of alterations in cfDNA were derived from white blood cells. After filtering blood cell alterations from cfDNA, it was found that the presence of ctDNA can predict recurrence when analyzed after preoperative treatment (median 18.4 months vs. median not reached, P=0.012, HR=3.0, 95% CI=1.3-6.9) and after surgery (median 18.7 months vs. median not reached, P<0.001, HR=21.8, 95% CI=3.9-123.1) in patients eligible for multimodal treatment. These analyses provide a facile method for distinguishing ctDNA from other cfDNA alterations, and highlight the utility of ctDNA as a predictive biomarker of patient outcome to perioperative therapy and resection in patients with, for example, gastric cancer, as well as other cancers including colorectal cancer, lung cancer, and/or esophageal cancer.

Candidate tumor-specific mutations in cfDNA, consisting of point mutations, small insertions, and deletions can be identified across the targeted regions of interest as described in detail in the examples section which follows. Briefly, an alteration was considered a candidate somatic mutation only when: (i) Three distinct paired reads contained the mutation in the cf-DNA and the number of distinct paired reads containing a particular mutation in the plasma was at least 0.05% of the total distinct read pairs; or (ii) one distinct paired read contained the mutation in the cfDNA and the mutation had also been detected in at least one additional timepoint at the level specified in (i); (iii) the mismatched base or small indel was not identified in matched white blood cell sequencing data of samples collected at baseline at the level of one distinct read (Table 9); (iv) the mismatched base or small indel was not present in a custom database of common germline variants derived from dbSNP; (v) the altered base did not arise from misplaced genome alignments including paralogous sequences; and (vi) the mutation fell within a protein coding region and was classified as a missense, nonsense, frameshift, or splice site alteration. Candidate alterations were defined as somatic hotspots if the nucleotide change and amino acid change were identical to an alteration observed in >20 cancer cases reported in the COSMIC database.

### Genomes and Cancer

Cancer genome sequencing studies have collectively identified various genetic mutations that make human tumors grow and progress. Unlike hereditary or germline mutations that are passed from parent to child, somatic mutations form in the DNA of individual cells during a person's life and are not passed from parent to child. Therefore, sequence variants due to somatic DNA mutations that are associated with cancers provide biomarkers to detect cancers and measure development of cancers.

Tumor tissues per se include large amount of DNA materials that may be analyzed to detect cancer variants, or sequence variants that are known to or suspected to be associated with various cancers. This can be performed through biopsy of tumor tissues. However, due to the continuously changing location and form of cancers, it is often difficult to continuously obtain biopsy samples at various locations to obtain cancer tissues and cancer originating DNA. Dying tumor cells release small pieces of their DNA into the bloodstream and other bodily fluids. These pieces are called cell free circulating tumor DNA (ctDNA), which coexists with cell-free DNA (cfDNA) from non-cancer cells. Screenings of ctDNA related to somatic mutations detect and follow the progression of a patient's tumor. These methods are also referred to as liquid biopsy.

Various current liquid biopsy methods utilize high throughput sequencing to analyze cfDNA collected from patients. However, the ability to detect tumor-specific variants is bounded by several factors. Liquid biopsy methods utilizing high throughput sequencing are limited by sequencing error rate and sequencing depth. In some cancer patients, tumor load may be very load for some tumor variant. For instance, the ctDNA may be fewer than 0.1%, or 0.01% in some samples. So the fraction of cfDNA originating from tumors can fall below the margin of error of sequencing pipeline. Tumor-specific variants called from low tumor burden patients can be plagued by high false positive rates, because there is small but existing chance that a sequence matching the tumor variant in a putative read is in fact due to sequencing errors instead of an actual mutation. It is desirable to increase true positive to improve sensitivity and decrease false positive to improve selectivity.

Accordingly, in certain embodiments, a method of detecting tumor specific mutations in a subject's circulating tumor DNA, comprises obtaining a sample from a subject at risk or suffering from cancer. In certain embodiments, the sample is whole blood. The whole blood is processed, e.g. centrifuged to separate the plasm from the cellular components. cfDNA is then extracted from the plasma and genomic DNA is extracted, for example, from white blood cells. Sequencing libraries of genomic DNA comprising cell free DNA (cfDNA) and cellular DNA are prepared to identify sequence variations in the cfDNA and cellular DNA as compared to a reference genomic sequence. The sequence variations between the cfDNA and cellular DNA are compared to identify differences in the sequences. Sequence specific mutations detected in both cfDNA and white blood cell DNA were excluded as tumor specific mutations. Sequence specific mutations detected exclusively in cfDNA were identified as tumor specific mutations.

In certain embodiments, detection of mutations in circulating tumor DNA of subjects with cancer are determinative of eligibility for surgical resection.

In certain embodiments, detection of changes in levels of circulating tumor DNA in patients treated with perioperative chemotherapy is determinative of whether the patient is responding to the therapy. For example, a decrease in levels of circulating tumor DNA detected in patients treated with neoadjuvant anti-cancer agents.

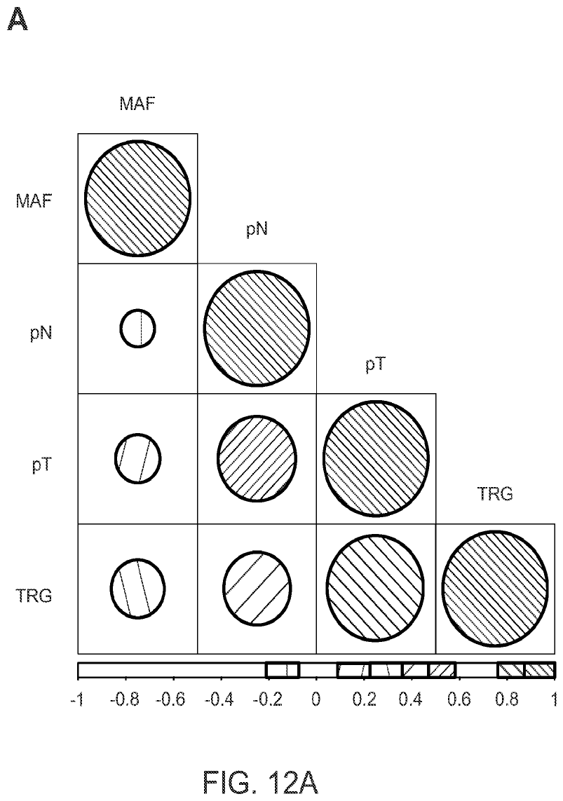

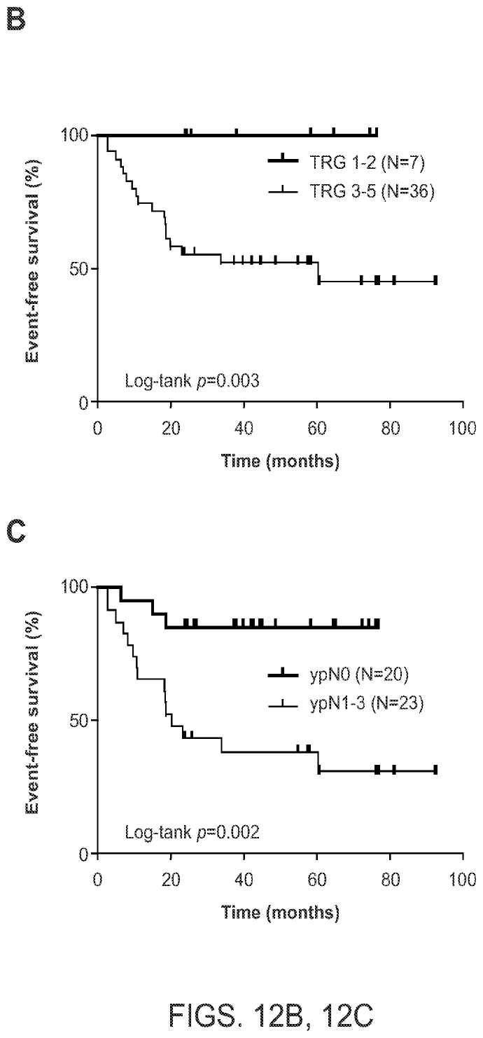

In certain embodiments, prediction of pathological response to preoperative chemotherapy in patients with cancer is determinative of whether the treatment is reacting negatively to the therapy. See, for example, FIGS. 12A-12C.

In certain embodiments, a decrease in levels of circulating tumor DNA or number and type of mutations detected is prediction of recurrence after perioperative treatment in patients with cancer.

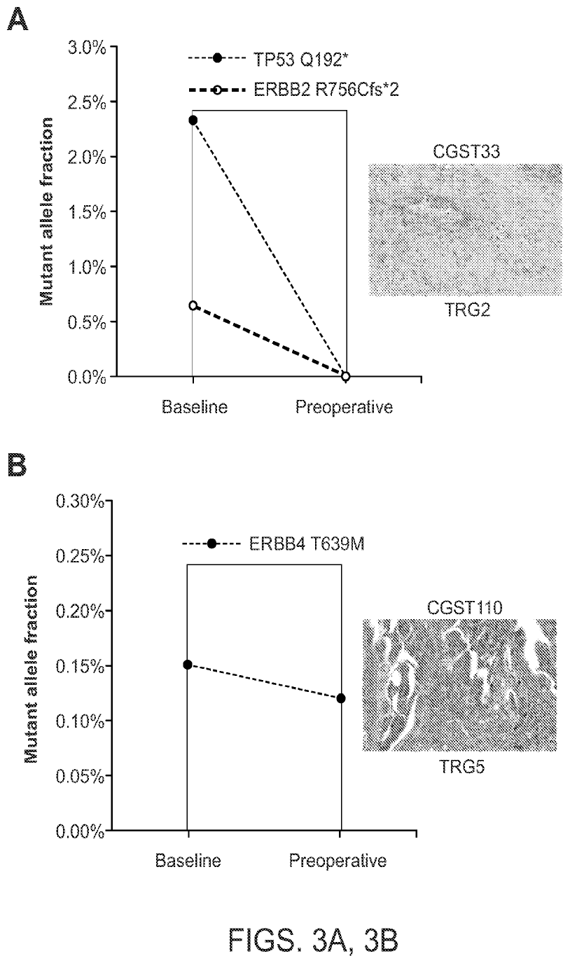

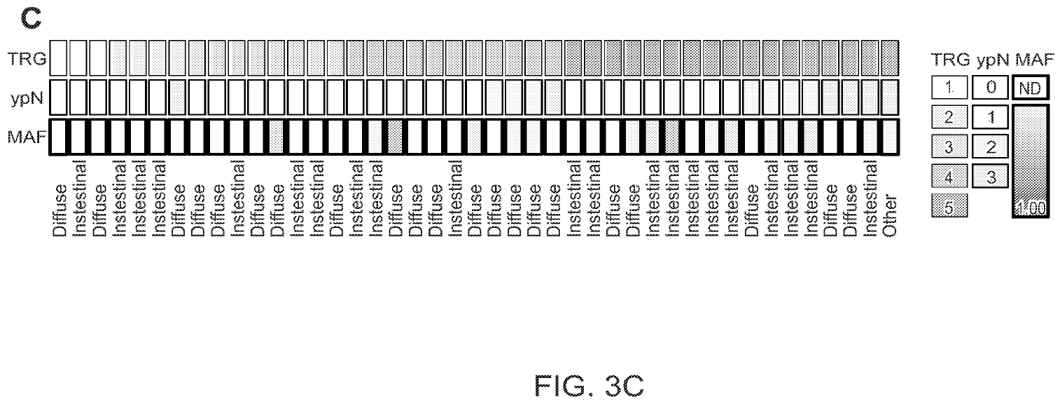

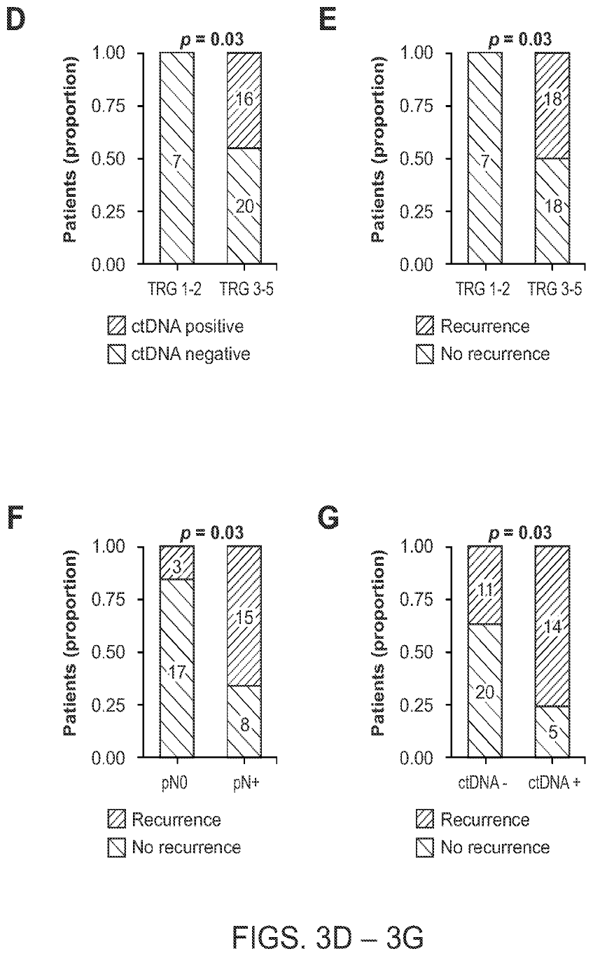

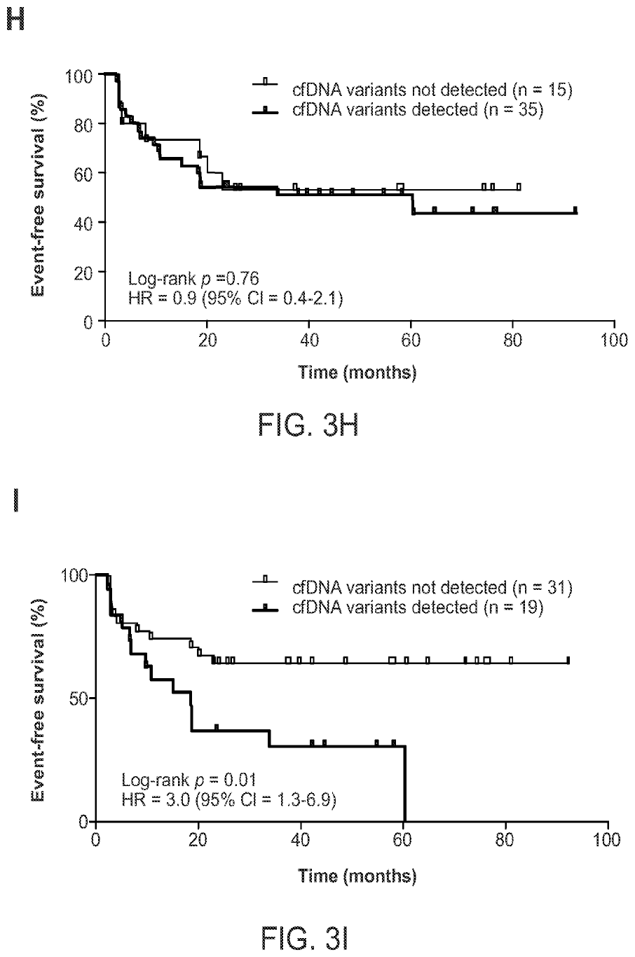

In certain embodiments, a change in levels of circulating tumor DNA or number and type of mutations detected is a prediction of cancer-specific survival after perioperative treatment in patients with cancer. For example a decrease in circulating tumor DNA, or a decrease in the number and types of mutations detected that are exclusive to cfDNA would be predictive of survival. See, for example FIGS. 3A-3I.

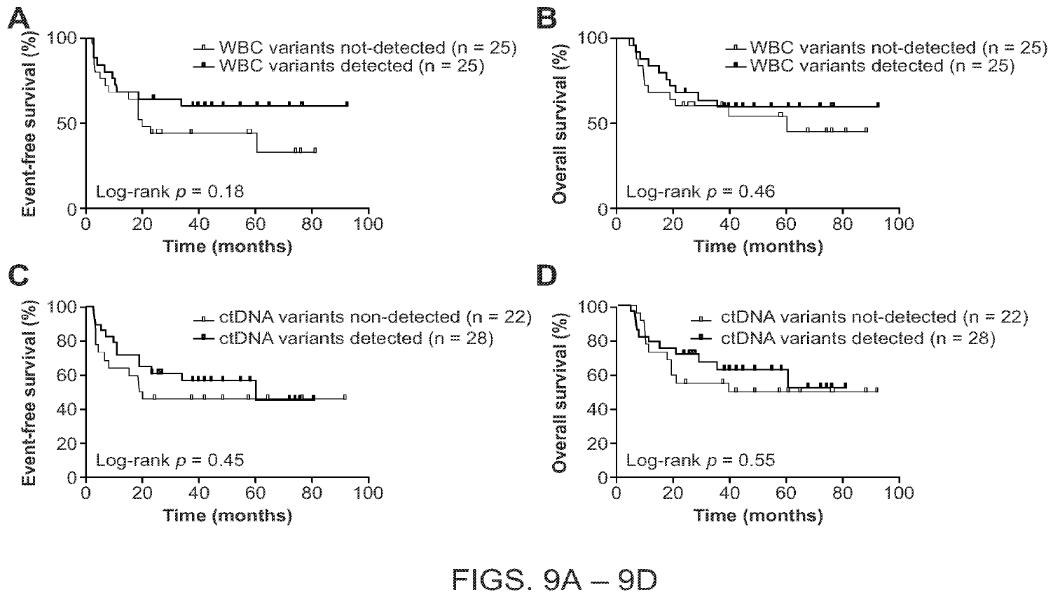

In certain embodiments, a change in levels of circulating tumor DNA or number and type of mutations detected is prediction of overall survival after perioperative treatment in patients with cancer. See, for example, FIGS. 9A-9D.

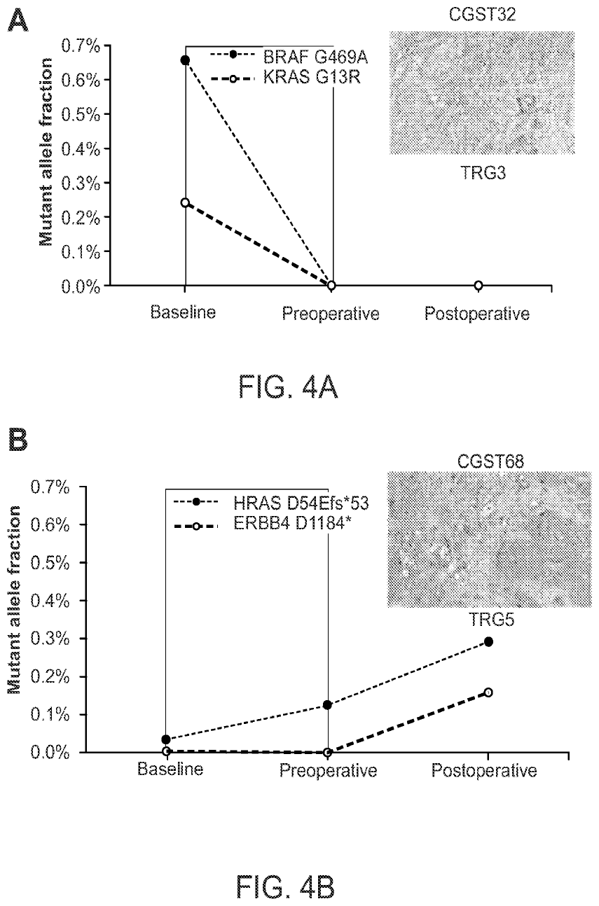

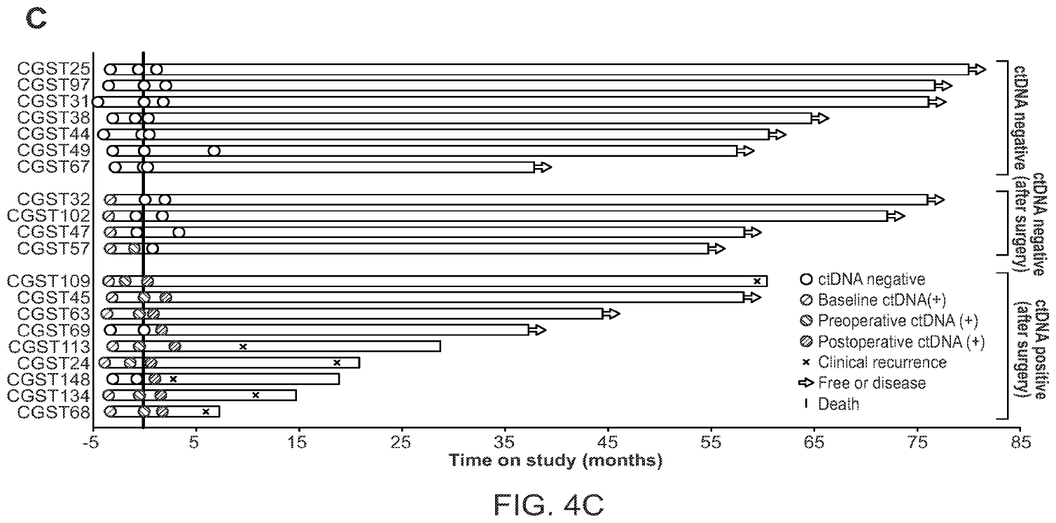

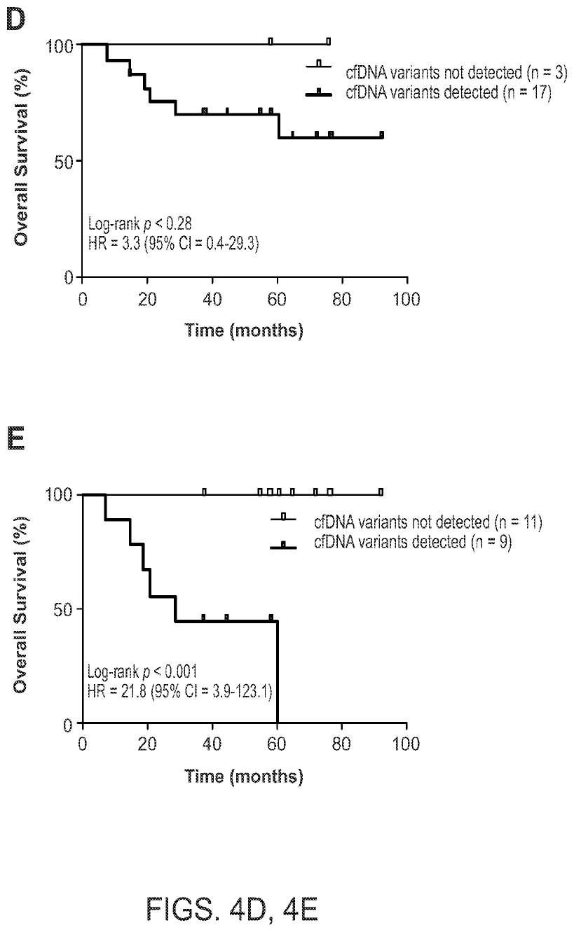

In certain embodiments, a change in levels of circulating tumor DNA or number and type of mutations detected is determinative of minimal residual disease after tumor resection in patients with cancer. See, for example, FIGS. 4A-4E.

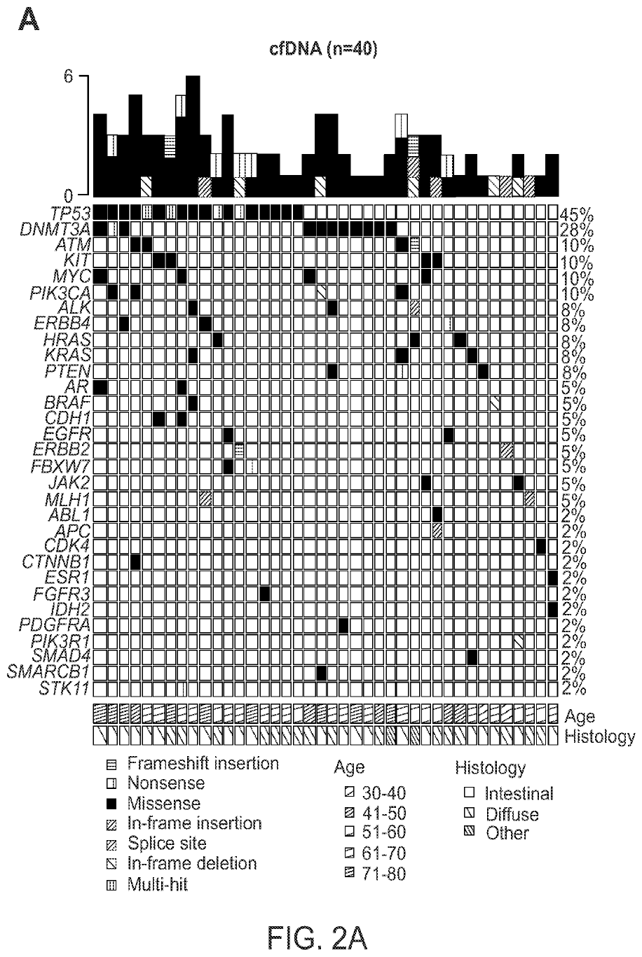

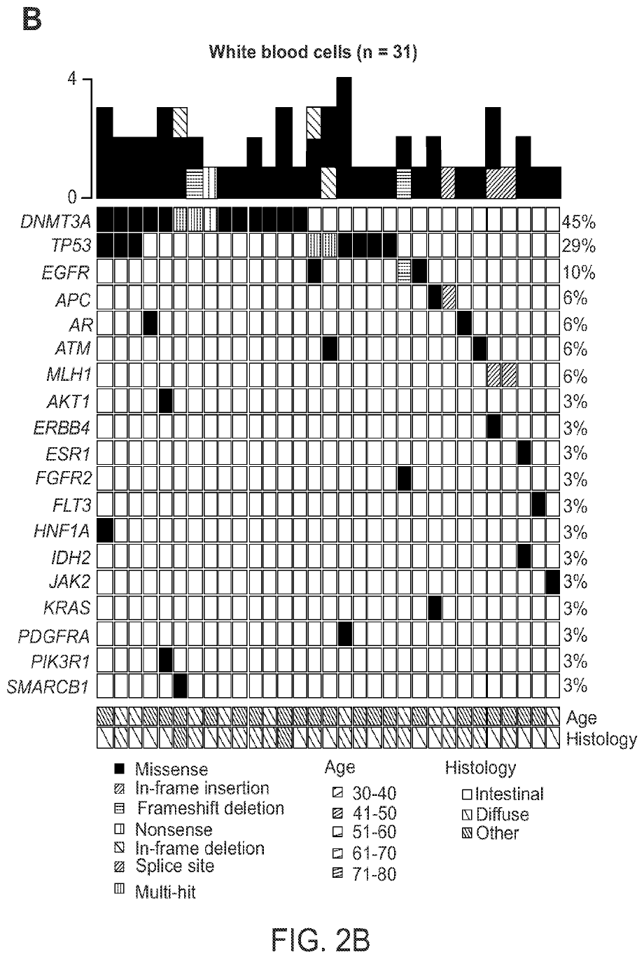

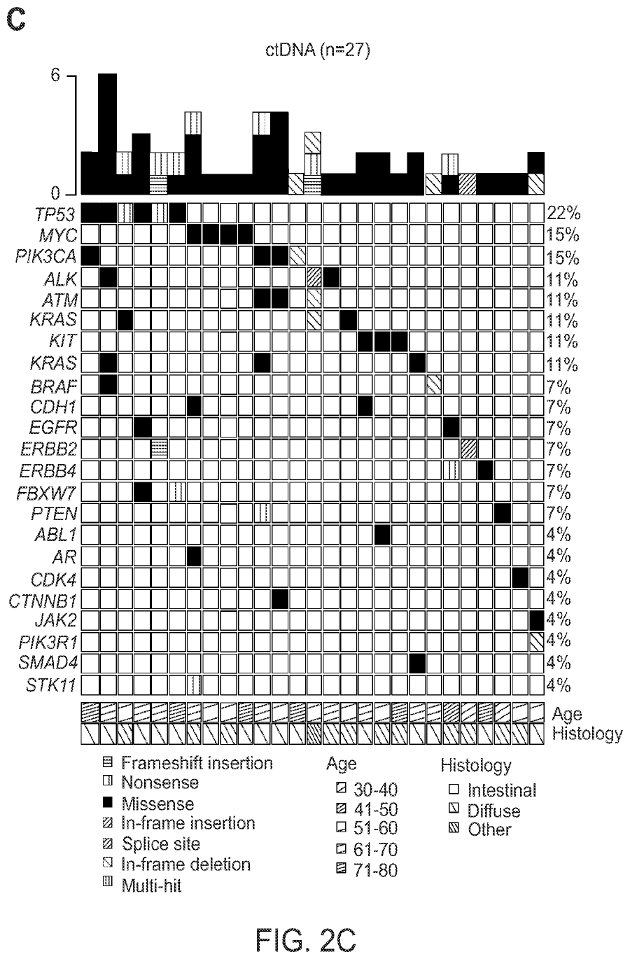

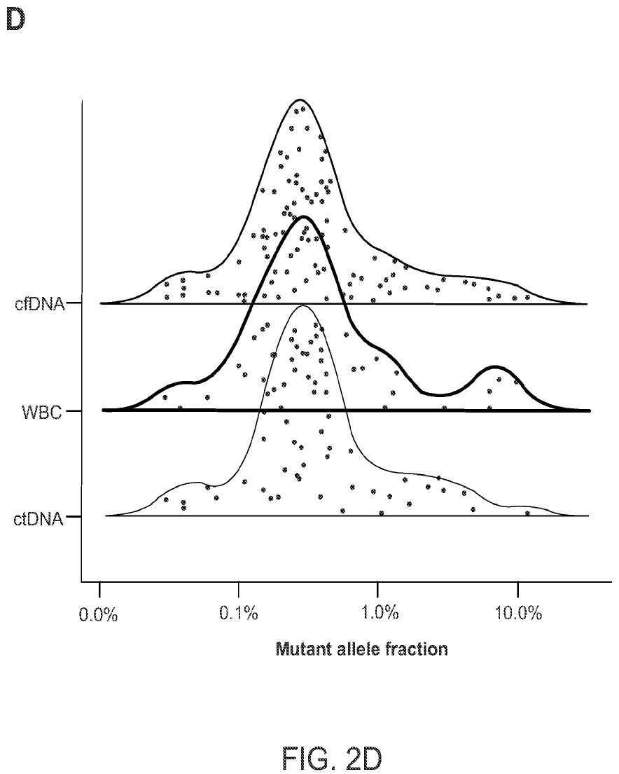

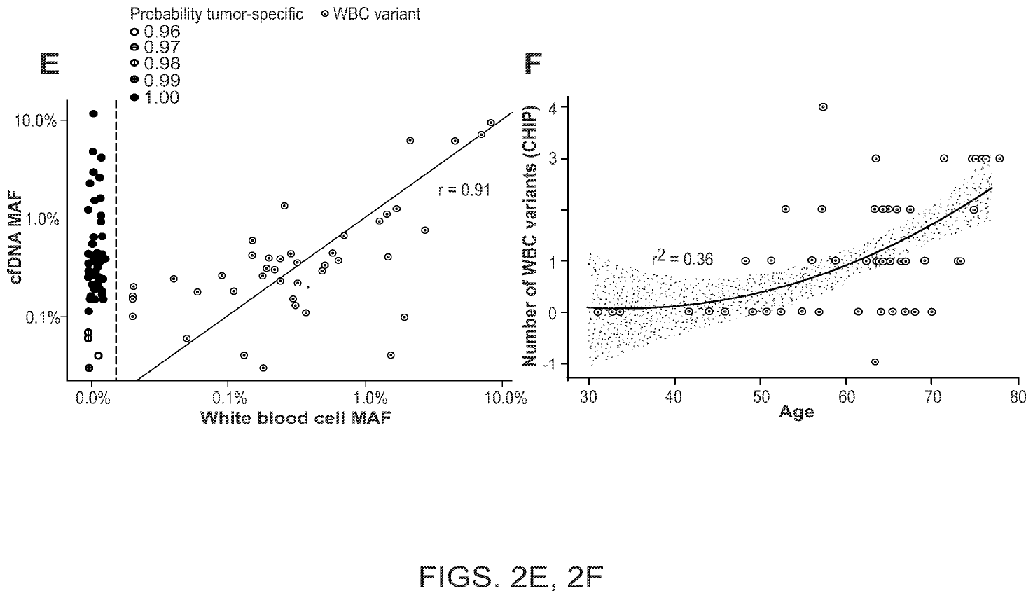

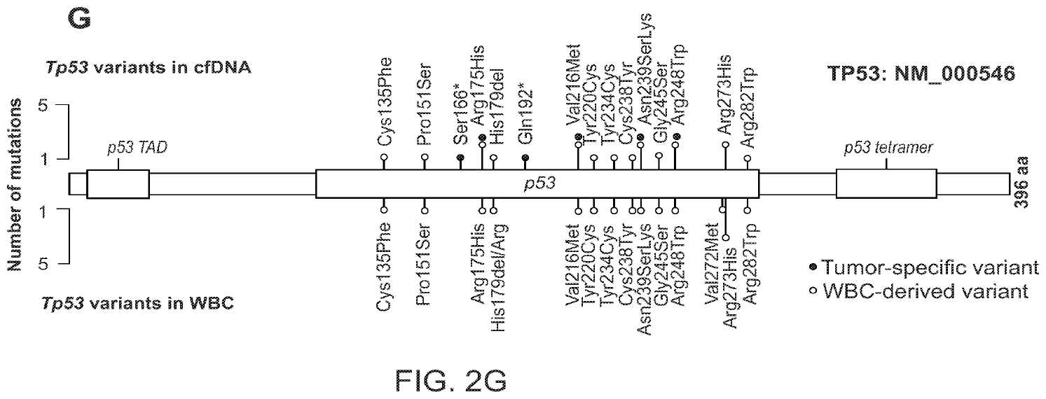

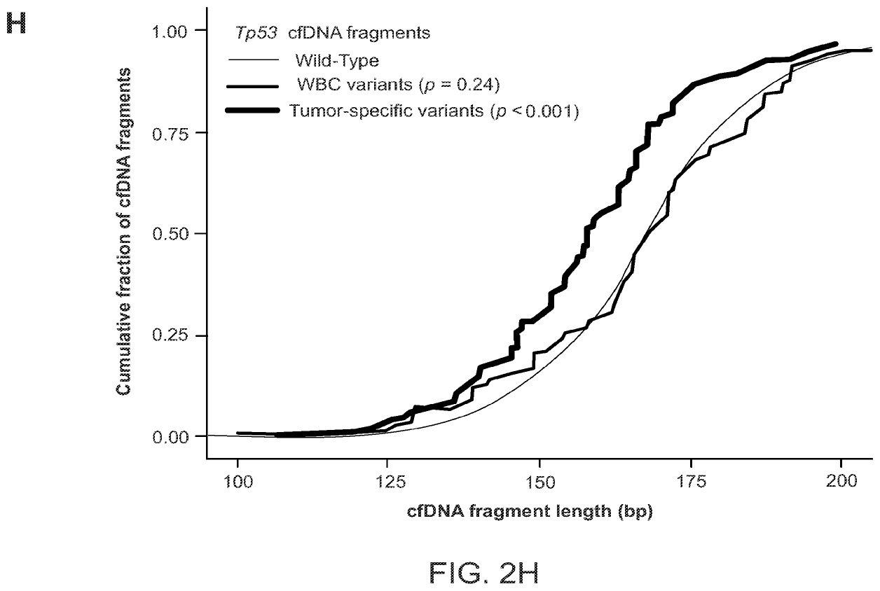

In certain embodiments, detection of alterations associated with clonal hematopoiesis in patients with cancer, is determinative of whether the subject is eligible for systemic treatment with anti-cancer agents. See, for example FIGS. 2A, 2B and Tables 6 and 7.

In certain embodiments, a change in levels of circulating tumor DNA or number and type of mutations detected, provides an identification of patients that will benefit from receiving neoadjuvant systemic treatment before tumor resection.

After purification of cfDNA from biological fluids, for example, using standard techniques, the fragments are subjected to one or more enzymatic steps to create a sequencing library. These enzymatic steps may include one or more of 5′ phosphorylation, end repair with a polymerase, A-tailing with a polymerase, ligation of one or more sequencing adapters with a ligase, and linear or exponential amplification of a plurality of fragments with a polymerase. In some embodiments, a plurality of fragments whose sequence composition matches a pre-defined panel of sequences may be targeted or selected by hybridization-capture, such that a subset of the starting library is carried forward for additional steps.

Amplification adapters may be attached to the fragmented nucleic acid. Adapters may be commercially obtained, such as from Integrated DNA Technologies (Coralville, Iowa). In certain embodiments, the adapter sequences are attached to the template nucleic acid molecule with an enzyme. The enzyme may be a ligase or a polymerase. The ligase may be any enzyme capable of ligating an oligonucleotide (RNA or DNA) to the template nucleic acid molecule. Suitable ligases include T4 DNA ligase and T4 RNA ligase, available commercially from New England Biolabs (Ipswich, Mass.). Methods for using ligases are well known in the art. The polymerase may be any enzyme capable of adding nucleotides to the 3′ and the 5′ terminus of template nucleic acid molecules.

The ligation may be blunt ended or utilize complementary overhanging ends. In certain embodiments, the ends of the fragments may be repaired, trimmed (e.g. using an exonuclease), or filled (e.g., using a polymerase and dNTPs) following fragmentation to form blunt ends. In some embodiments, end repair is performed to generate blunt end 5′ phosphorylated nucleic acid ends using commercial kits, such as those available from Epicentre Biotechnologies (Madison, Wis.). Upon generating blunt ends, the ends may be treated with a polymerase and dATP to form a template independent addition to the 3′-end and the 5′-end of the fragments, thus producing a single A overhanging. This single A is used to guide ligation of fragments with a single T overhanging from the 5′-end in a method referred to as T-A cloning. Alternatively, because the possible combinations of overhangs left by the restriction enzymes are known after a restriction digestion, the ends may be left as-is, i.e., ragged ends. In certain embodiments, double stranded oligonucleotides with complementary overhanging ends are used.

In certain embodiments, barcode sequences are attached to the template nucleic acids. In certain embodiments, a barcode is attached to each fragment. In other embodiments, a plurality of barcodes, e.g., two barcodes, are attached to each fragment. A barcode sequence generally includes certain features that make the sequence useful in sequencing reactions. For example the barcode sequences are designed to have minimal or no homo-polymer regions, i.e., 2 or more of the same base in a row such as AA or CCC, within the barcode sequence. The barcode sequences are also designed so that they are at least one edit distance away from the base addition order when performing base-by-base sequencing, ensuring that the first and last base do not match the expected bases of the sequence.

The barcode sequences are designed such that each sequence is correlated to a particular portion of nucleic acid, allowing sequence reads to be correlated back to the portion from which they came. In certain embodiments, the barcode sequences range from about 5 nucleotides to about 15 nucleotides. In a particular embodiment, the barcode sequences range from about 4 nucleotides to about 7 nucleotides. Since the barcode sequence is sequenced along with the template nucleic acid, the oligonucleotide length should be of minimal length so as to permit the longest read from the template nucleic acid attached. For example, a plurality of DNA barcodes can comprise various numbers of sequences of nucleotides. In certain embodiments, the barcode sequences comprise 2, 3, 4, 5, 6, 7, 8, 9, 10, 11, 12, 13, 14, 15, 16, 17, 18, 19, 20, 21, 22, 23, 24, 25, 26, 27, 28, 29, 30 or more nucleotides. When attached to only one end of a polynucleotide, the plurality of DNA barcodes can produce 2, 3, 4, 5, 6, 7, 8, 9, 10, 11, 12, 13, 14, 15, 16, 17, 18, 19, 20, 21, 22, 23, 24, 25, 26, 27, 28, 29, 30 or more different identifiers. Alternatively, when attached to both ends of a polynucleotide, the plurality DNA barcodes can produce 4, 9, 16, 25, 36, 49, 64, 81, 100, 121, 144, 169, 196, 225, 256, 289, 324, 361, 400 or more different identifiers (which is the 2 of when the DNA barcode is attached to only 1 end of a polynucleotide).

Generally, the barcode sequences are spaced from the template nucleic acid molecule by at least one base (minimizes homo-polymeric combinations). In certain embodiments, the barcode sequences are attached to the template nucleic acid molecule, e.g., with an enzyme. The enzyme may be a ligase or a polymerase, as discussed below.

Amplification or sequencing adapters or barcodes, or a combination thereof, may be attached to the fragmented nucleic acid. Such molecules may be commercially obtained, such as from Integrated DNA Technologies (Coralville, Iowa). In certain embodiments, such sequences are attached to the template nucleic acid molecule with an enzyme such as a ligase. Suitable ligases include T4 DNA ligase and T4 RNA ligase, available commercially from New England Biolabs (Ipswich, Mass.). The ligation may be blunt ended or via use of complementary overhanging ends. In certain embodiments, following fragmentation, the ends of the fragments may be repaired, trimmed (e.g. using an exonuclease), or filled (e.g., using a polymerase and dNTPs) to form blunt ends. In some embodiments, end repair is performed to generate blunt end 5′ phosphorylated nucleic acid ends using commercial kits, such as those available from Epicentre Biotechnologies (Madison, Wis.). Upon generating blunt ends, the ends may be treated with a polymerase and dATP to form a template independent addition to the 3′-end and the 5′-end of the fragments, thus producing a single A overhanging. This single A can guide ligation of fragments with a single T overhanging from the 5′-end in a method referred to as T-A cloning. Alternatively, because the possible combinations of overhangs left by the restriction enzymes are known after a restriction digestion, the ends may be left as-is, i.e., ragged ends. In certain embodiments double stranded oligonucleotides with complementary overhanging ends are used.

After any processing steps (e.g., obtaining, isolating, fragmenting, amplification, or barcoding), nucleic acid can be sequenced.

Sequencing: In certain embodiments, a high-throughput sequencing method is used. In certain embodiments, a next generation sequencing method is used. See, for example, Phallen, J. et al. Direct detection of early-stage cancers using circulating tumor DNA. Sci Transl Med 9, doi:10.1126/scitranslmed.aan2415 (2017). Li B. T. et al. Annals of Oncology 30: 597-603, 2019, doi:10.1093/annonc/mdz04, each of which are incorporated by reference in their entirety. Non-limiting examples of NGS include sequencing-by-synthesis using reversible dye terminators, and sequencing-by-ligation. This method is based on targeted capture and deep sequencing (>30,000×) of DNA fragments to identify single base substitutions and small insertions or deletions in cfDNA across 80,930 bp of coding gene regions while distinguishing these from PCR amplification and sequencing artifacts.

Sequencing may also be by any method known in the art. DNA sequencing techniques include classic dideoxy sequencing reactions (Sanger method) using labeled terminators or primers and gel separation in slab or capillary, sequencing by synthesis using reversibly terminated labeled nucleotides, pyrosequencing, 454 sequencing, Illumina/Solexa sequencing, allele specific hybridization to a library of labeled oligonucleotide probes, sequencing by synthesis using allele specific hybridization to a library of labeled clones that is followed by ligation, real time monitoring of the incorporation of labeled nucleotides during a polymerization step, polony sequencing, SOLiD sequencing targeted sequencing, single molecule real-time sequencing, exon sequencing, electron microscopy-based sequencing, panel sequencing, transistor-mediated sequencing, direct sequencing, random shotgun sequencing, whole-genome sequencing, sequencing by hybridization, capillary electrophoresis, gel electrophoresis, duplex sequencing, cycle sequencing, single-base extension sequencing, solid-phase sequencing, high-throughput sequencing, massively parallel signature sequencing, emulsion PCR, co-amplification at lower denaturation temperature-PCR (COLD-PCR), multiplex PCR, sequencing by reversible dye terminator, paired-end sequencing, near-term sequencing, exonuclease sequencing, sequencing by ligation, short-read sequencing, single-molecule sequencing, real-time sequencing, reverse-terminator sequencing, nanopore sequencing, MS-PET sequencing, and a combination thereof. In some embodiments, the sequencing method is massively parallel sequencing, that is, simultaneously (or in rapid succession) sequencing any of at least 100, 1000, 10,000, 100,000, 1 million, 10 million, 100 million, or 1 billion polynucleotide molecules. In some embodiments, sequencing can be performed by a gene analyzer such as, for example, gene analyzers commercially available from Illumina or Applied Biosystems. Sequencing of separated molecules has more recently been demonstrated by sequential or single extension reactions using polymerases or ligases as well as by single or sequential differential hybridizations with libraries of probes. Sequencing may be performed by a DNA sequencer (e.g., a machine designed to perform sequencing reactions).

A sequencing technique that can be used includes, for example, use of sequencing-by-synthesis systems. In the first step, DNA is sheared into fragments of approximately 300-800 base pairs, and the fragments are blunt ended. Oligonucleotide adaptors are then ligated to the ends of the fragments. The adaptors serve as primers for amplification and sequencing of the fragments. The fragments can be attached to DNA capture beads, e.g., streptavidin-coated beads using, e.g., Adaptor B, which contains 5′-biotin tag. The fragments attached to the beads are PCR amplified within droplets of an oil-water emulsion. The result is multiple copies of clonally amplified DNA fragments on each bead. In the second step, the beads are captured in wells (pico-liter sized). Pyrosequencing is performed on each DNA fragment in parallel. Addition of one or more nucleotides generates a light signal that is recorded by a CCD camera in a sequencing instrument. The signal strength is proportional to the number of nucleotides incorporated. Pyrosequencing makes use of pyrophosphate (PPi) which is released upon nucleotide addition. PPi is converted to ATP by ATP sulfurylase in the presence of adenosine 5′ phosphosulfate. Luciferase uses ATP to convert luciferin to oxyluciferin, and this reaction generates light that is detected and analyzed.

Another example of a DNA sequencing technique that can be used is SOLiD™ technology by Applied Biosystems from Life Technologies Corporation (Carlsbad, Calif.). In SOLiD™ sequencing, genomic DNA is sheared into fragments, and adaptors are attached to the 5′ and 3′ ends of the fragments to generate a fragment library. Alternatively, internal adaptors can be introduced by ligating adaptors to the 5′ and 3′ ends of the fragments, circularizing the fragments, digesting the circularized fragment to generate an internal adaptor, and attaching adaptors to the 5′ and 3′ ends of the resulting fragments to generate a mate-paired library. Next, clonal bead populations are prepared in microreactors containing beads, primers, template, and PCR components. Following PCR, the templates are denatured and beads are enriched to separate the beads with extended templates. Templates on the selected beads are subjected to a 3′ modification that permits bonding to a glass slide. The sequence can be determined by sequential hybridization and ligation of partially random oligonucleotides with a central determined base (or pair of bases) that is identified by a specific fluorophore. After a color is recorded, the ligated oligonucleotide is removed and the process is then repeated.

Another example of a DNA sequencing technique that can be used is ion semiconductor sequencing using, for example, a system sold under the trademark ION TORRENT by Ion Torrent by Life Technologies (South San Francisco, Calif.). Ion semiconductor sequencing is described, for example, in Rothberg, et al., An integrated semiconductor device enabling non-optical genome sequencing, Nature 475:348-352 (2011); U.S. Pub. 2010/0304982; U.S. Pub. 2010/0301398; U.S. Pub. 2010/0300895; U.S. Pub. 2010/0300559; and U.S. Pub. 2009/0026082, the contents of each of which are incorporated by reference in their entirety.

Another example of a sequencing technology that can be used is Illumina sequencing. Illumina sequencing is based on the amplification of DNA on a solid surface using fold-back PCR and anchored primers. Genomic DNA is fragmented, and adapters are added to the 5′ and 3′ ends of the fragments. DNA fragments that are attached to the surface of flow cell channels are extended and bridge amplified. The fragments become double stranded, and the double stranded molecules are denatured. Multiple cycles of the solid-phase amplification followed by denaturation can create several million clusters of approximately 1,000 copies of single-stranded DNA molecules of the same template in each channel of the flow cell. Primers, DNA polymerase and four fluorophore-labeled, reversibly terminating nucleotides are used to perform sequential sequencing. After nucleotide incorporation, a laser is used to excite the fluorophores, and an image is captured and the identity of the first base is recorded. The 3′ terminators and fluorophores from each incorporated base are removed and the incorporation, detection and identification steps are repeated. Sequencing according to this technology is described in U.S. Pat. Nos. 7,960,120; 7,835,871; 7,232,656; 7,598,035; 6,911,345; 6,833,246; 6,828,100; 6,306,597; 6,210,891; U.S. Pub. 2011/0009278; U.S. Pub. 2007/0114362; U.S. Pub. 2006/0292611; and U.S. Pub. 2006/0024681, each of which are incorporated by reference in their entirety.

Another example of a sequencing technology that can be used includes the single molecule, real-time (SMRT) technology of Pacific Biosciences (Menlo Park, Calif.). In SMRT, each of the four DNA bases is attached to one of four different fluorescent dyes. These dyes are phospholinked. A single DNA polymerase is immobilized with a single molecule of template single stranded DNA at the bottom of a zero-mode waveguide (ZMW). It takes several milliseconds to incorporate a nucleotide into a growing strand. During this time, the fluorescent label is excited and produces a fluorescent signal, and the fluorescent tag is cleaved off. Detection of the corresponding fluorescence of the dye indicates which base was incorporated. The process is repeated.

Another example of a sequencing technique that can be used is nanopore sequencing (Soni & Meller, 2007, Progress toward ultrafast DNA sequence using solid-state nanopores, Clin Chem 53(11):1996-2001). A nanopore is a small hole, of the order of 1 nanometer in diameter. Immersion of a nanopore in a conducting fluid and application of a potential across it results in a slight electrical current due to conduction of ions through the nanopore. The amount of current which flows is sensitive to the size of the nanopore. As a DNA molecule passes through a nanopore, each nucleotide on the DNA molecule obstructs the nanopore to a different degree. Thus, the change in the current passing through the nanopore as the DNA molecule passes through the nanopore represents a reading of the DNA sequence.

Another example of a sequencing technique that can be used involves using a chemical-sensitive field effect transistor (chemFET) array to sequence DNA (for example, as described in U.S. Pub. 2009/0026082). In one example of the technique, DNA molecules can be placed into reaction chambers, and the template molecules can be hybridized to a sequencing primer bound to a polymerase. Incorporation of one or more triphosphates into a new nucleic acid strand at the 3′ end of the sequencing primer can be detected by a change in current by a chemFET. An array can have multiple chemFET sensors. In another example, single nucleic acids can be attached to beads, and the nucleic acids can be amplified on the bead, and the individual beads can be transferred to individual reaction chambers on a chemFET array, with each chamber having a chemFET sensor, and the nucleic acids can be sequenced.

Another example of a sequencing technique that can be used involves using an electron microscope as described, for example, by Moudrianakis, E. N. and Beer M., in Base sequence determination in nucleic acids with the electron microscope, III. Chemistry and microscopy of guanine-labeled DNA, PNAS 53:564-71 (1965). In one example of the technique, individual DNA molecules are labeled using metallic labels that are distinguishable using an electron microscope. These molecules are then stretched on a flat surface and imaged using an electron microscope to measure sequences.

Sequence Reads: Sequencing generates a plurality of reads. Reads generally include sequences of nucleotide data less than about 150 bases in length, or less than about 90 bases in length. In certain embodiments, reads are between about 80 and about 90 bases, e.g., about 85 bases in length. In some embodiments, methods of the invention are applied to very short reads, i.e., less than about 50 or about 30 bases in length. Sequence read data can include the sequence data as well as meta information. Sequence read data can be stored in any suitable file format including, for example, VCF files, FASTA files or FASTQ files, as are known to those of skill in the art.

FASTA is originally a computer program for searching sequence databases and the name FASTA has come to also refer to a standard file format. See Pearson & Lipman, 1988, Improved tools for biological sequence comparison, PNAS 85:2444-2448. The FASTQ format is a text-based format for storing both a biological sequence (usually nucleotide sequence) and its corresponding quality scores. It is similar to the FASTA format but with quality scores following the sequence data. Both the sequence letter and quality score are encoded with a single ASCII character for brevity. The FASTQ format is a de facto standard for storing the output of high throughput sequencing instruments such as the Illumina Genome Analyzer. Cock et al., 2009, The Sanger FASTQ file format for sequences with quality scores, and the Solexa/Illumina FASTQ variants, Nucleic Acids Res 38(6):1767-1771.

Certain embodiments of the invention provide for the assembly of sequence reads. In assembly by alignment, for example, the reads are aligned to each other or to a reference. By aligning each read, in turn to a reference genome, all of the reads are positioned in relationship to each other to create the assembly. In addition, aligning or mapping the sequence read to a reference sequence can also be used to identify variant sequences within the sequence read. Identifying variant sequences can be used in combination with the methods and systems described herein to further aid in the diagnosis or prognosis of a disease or condition, or for guiding treatment decisions.

In certain embodiments, sequence reads are aligned against the human reference genome (hg19) with additional realignment of select regions. Candidate tumor-specific mutations in cfDNA, consisting of point mutations, small insertions, and deletions are identified using across the targeted regions of interest. Candidate alterations are defined as somatic hotspots if the nucleotide change and amino acid change are identical to an alteration observed in ≥20 cancer cases reported in the COSMIC database.

For alterations detected in cfDNA that were not identified by matched white blood cell sequencing, the posterior probability that such an alteration was tumor derived was determined from a Bayesian statistical model using the frequency of altered alleles and total coverage of cfDNA and WBCs sequences.

A processing system, such as a processor of a computer, is used for executing the code for performing the variant computational analysis. Analyses of groups of mutations correlation coefficients are determined for the association between WBC variants and their correspondent alterations identified in cfDNA, as well as for the association between the number of WBC variants and age.

For mutations identified by cfDNA sequencing but not identified by WBC sequencing, the probability for the model that the mutation is tumor derived relative to the probability for the model that the mutation was hematopoietic is computed. The sampling distribution of the observed number of reads with an altered mutation in cfDNA and WBC sequencing is a binomial parameterized by the total coverage at that mutation and unknown probability theta. This method is described in detail in the examples section which follows.

In some embodiments, the processing system uses one or more different types of models. For example, a Bayesian hierarchical model is one of many possible model architectures that may be used to generate candidate variants. Further, multiple different models may be stored in a database or retrieved for application post-training. For example, a first model is trained to model single nucleotide variants (SNV) noise rates and a second model is trained to model insertion deletion noise rates. Further, the processing system may use parameters of the model to determine a likelihood of one or more true positives in a sequence read. The processing system may determine a quality score (e.g., on a logarithmic scale) based on the likelihood. Other models, such as a joint model, may use output of one or more Bayesian hierarchical models to determine expected noise of nucleotide mutations in sequence reads of different samples.

In some embodiments, any or all of the steps of the invention are automated. Alternatively, methods of the invention may be embodied wholly or partially in one or more dedicated programs, for example, each optionally written in a compiled language such as C++ then compiled and distributed as a binary. Methods of the invention may be implemented wholly or in part as modules within, or by invoking functionality within, existing sequence analysis platforms. In certain embodiments, methods of the invention include a number of steps that are all invoked automatically responsive to a single starting queue (e.g., one or a combination of triggering events sourced from human activity, another computer program, or a machine). Thus, the invention provides methods in which any or the steps or any combination of the steps can occur automatically responsive to a queue. Automatically generally means without intervening human input, influence, or interaction (i.e., responsive only to original or pre-queue human activity).

In some embodiments of any of the systems provided herein, the sequencer is configured to perform next generation sequencing (NGS). In some embodiments, the sequencer is configured to perform massively parallel sequencing using sequencing-by-synthesis with reversible dye terminators. In other embodiments, the sequencer is configured to perform sequencing-by-ligation. In yet other embodiments, the sequencer is configured to perform single molecule sequencing.

### EXAMPLES

### Example 1: Matched White Blood Cell and Cell-free DNA Analyses for Prediction of Therapeutic Response in Patients with Cancer

In the present study, a matched cfDNA and WBC sequencing approach was applied to accurately detect ctDNA alterations after preoperative chemotherapy and after surgery in patients with resectable gastric cancer. It was hypothesized that ctDNA detection after completion of preoperative treatment as well as minimal residual disease detection after surgery can predict recurrence and survival in patients with resectable gastric cancer treated with multimodal therapeutic regimens. Overall, these analyses evaluated a new strategy to distinguish ctDNA alterations from cfDNA variants related to clonal hematopoiesis and investigated whether ctDNA elimination before or after surgery can serve as a predictive biomarker of patient outcome to perioperative treatment.

### Materials and Methods

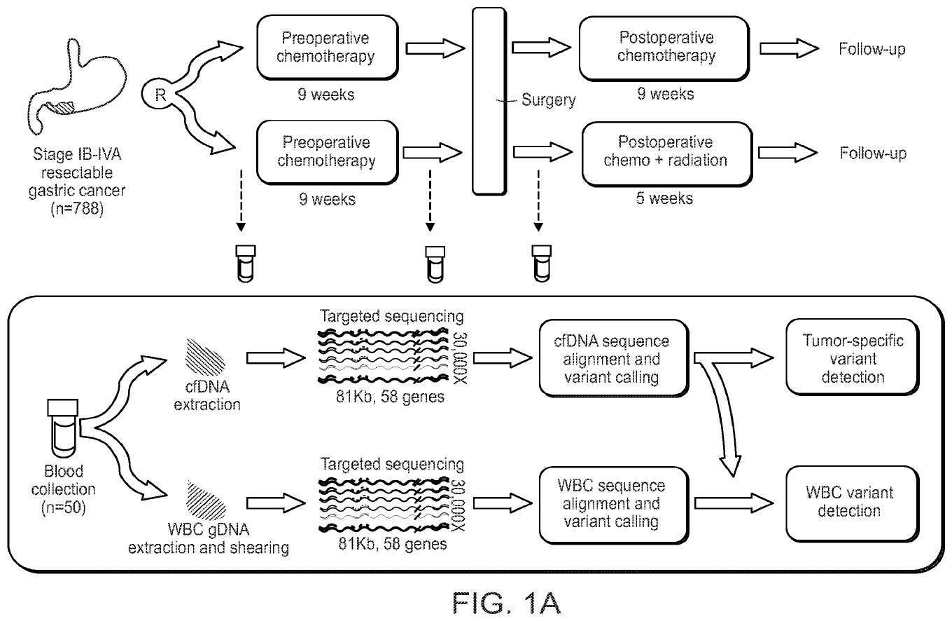

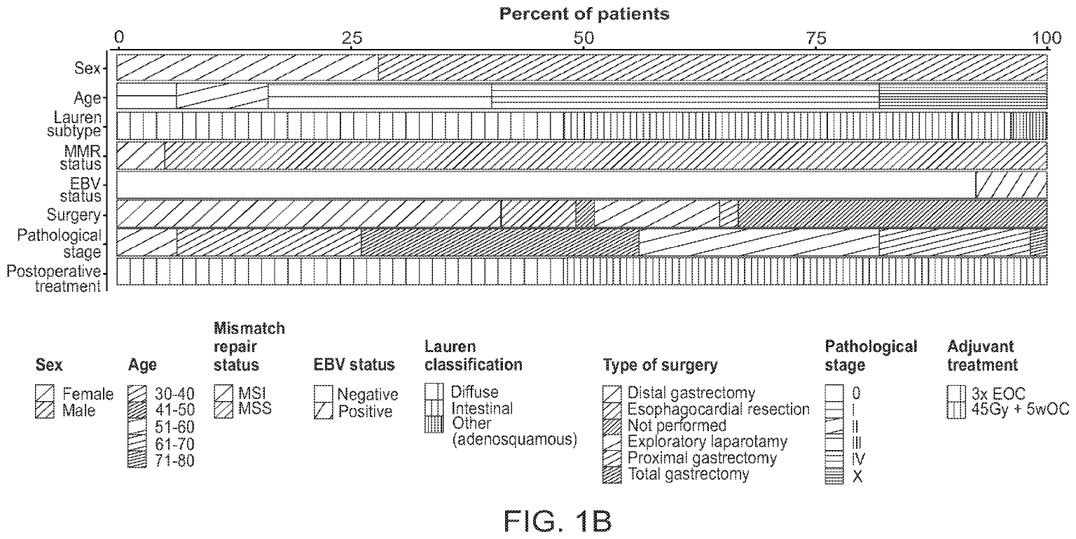

Experimental study design: The current study is a planned exploratory analysis of the predictive value of cfDNA assessment in 50 randomly selected patients from the CRITICS study (NCT00407186) who had plasma samples available and suitable for genomic analyses from at least two timepoints (FIG. 1A; FIG. 5; Table 1). The CRITICS study is an investigator-initiated, open-label, multi-center, phase III randomized controlled trial of perioperative chemotherapy (chemotherapy group) versus preoperative chemotherapy with postoperative chemo-radiotherapy (chemoradiotherapy group) in patients with resectable lung cancer (Cats A. et al. Lancet Oncol 19, 616-628, doi:10.1016/S1470-2045(18)30132-3 (2018)). A total of 788 patients from 56 hospitals in the Netherlands, Sweden, and Denmark were randomized upfront to receive three preoperative 21-day cycles of intravenous epirubicin, cisplatin or oxaliplatin, and oral capecitabine followed by three postoperative cycles of intravenous epirubicin, cisplatin or oxaliplatin, and oral capecitabine (chemotherapy group) or to receive the same preoperative regimen followed by postoperative radiotherapy combined with daily capecitabine and weekly cisplatin (chemoradiotherapy group) (FIG. 1A; FIG. 5; Table 1). Baseline blood samples at the time of trial enrollment were used for both cfDNA and white blood cell targeted deep sequencing (30,000×), followed by independent variant calling and further tumor-specific mutation detection using the white blood cell filtering approach (FIG. 1A). Tumor-specific mutations from the consecutive timepoints were identified using the white blood cell sequencing data from the same patient at baseline.

Patients and characteristics: Patients were eligible for the study if they had histologically proven gastric adenocarcinoma (as defined by the American Joint Committee on Cancer, 6th edition), stage TB-IVA (Greene, F. L. et al. American Joint Committee on Cancer: AJCC cancer staging manual. 6^{th} Ed., (Springer, New York, N.Y., 2002)), as assessed by esophagogastroduodenoscopy and CT of the chest, abdomen, and pelvis. Patients with tumors of the gastroesophageal junction were permitted to enroll when the bulk of the tumor was predominantly located in the stomach and could therefore consist of Siewert types II (true gastroesophageal junction) and III (subcardial stomach) tumors. Patients with Siewert type I (distal esophagus) tumors were not eligible. An exploratory laparoscopy was indicated when the preoperative CT scan suggested peritoneal carcinomatosis. Patient enrollment and genomic studies were conducted in accordance with the Declaration of Helsinki, were approved by the Institutional Review Board (IRB) and all patients provided written informed consent for sample acquisition for research purposes.

Pathological assessment of response, mismatch repair status and EBV status determination: Pathology slides from the resection specimen from each patient were collected and centrally reviewed by NCTvG to confirm histologic subtypes according to the Lauren's classification criteria (Lauren, P. The Two Histological Main Types of Gastric Carcinoma: Diffuse and So-Called Intestinal-Type Carcinoma. An Attempt at a Histo-Clinical Classification. Acta pathologica et microbiologica Scandinavica 64, 31-49 (1965)). Histopathological regression was determined by NCTvG according to Mandard's tumor regression grade (TRG) system: i) TRG1, no residual tumor left (pathological complete response); ii) TRG2, scattered tumor cells left; iii) TRG3, fibrosis outgrows tumor; iv) TRG4, tumor outgrows fibrosis; and v) TRG5, no histological signs of regression (Table 1). For detection of Epstein-Barr virus (EBV), the tumor areas were demarcated on H&E slides of the resection specimens. In case of sufficient amount of tumor tissue, 3 cores per tumor were taken for construction of a tissue microarray (TMA). TMA sections were cut and used for Epstein-Barr virus encoded RNA in-situ hybridization (EBER-ISH). In case little or no tumor was left in the resection specimen due to chemotherapy-induced pathological (near) complete response EBER-ISH was performed on the diagnostic biopsy specimen. EBER-ISH was performed using the U INFORM iViEW Blue ISH (v1.02.0023) and the INFORM EBER probe on the Benchmark Ultra IHC/ISH staining module (Roche Diagnostics, the Netherlands) according to the manufacturer's protocol (Table 1).

Formalin-fixed paraffin-embedded (FFPE) tissue blocks from the diagnostic biopsy specimen were used for MSI analysis. The tumor area was demarcated on an H&E slide. DNA was isolated from the demarcated tumor area. MSI analysis was performed using the MSI Analysis System (MSI Multiplex System Version 1.2, Promega) consisting of five nearly monomorphic mononucleotide markers (BAT-25, BAT-26, NR-21, NR-24, MONO-27) according to the manufacturer's instructions. PCR products were separated by capillary electrophoresis using an ABI 3500 Genetic Analyzer (Applied Biosystems, Foster City, Calif., USA), and analyzed using GeneMapper Software (Applied Biosystems, Foster City, Calif., USA). An internal lane size standard was added to the PCR samples for accurate sizing of alleles and to adjust for run-to run variations. When all markers were stable, the tumor was interpreted as microsatellite stable (MSS). The tumor was interpreted as MSI-low (MSI-L) if one marker was unstable and MSI-high (MSI-H) if two or more markers showed instability. MSI-L tumors were included in the MSS category (Table 1).

Sample preparation and next-generation sequencing of cfDNA and genomic DNA from white blood cells: Whole blood was collected in K2EDTA tubes, sent to the central pathology lab at VUmc, Amsterdam, and processed within 1 day after collection. Plasma and cellular components were separated by centrifugation at 1,300 rpm for 5 minutes in 1.5 ml microcentrifuge tubes at 4° C. and therefore stored at −20° C. until the time of DNA extraction. cfDNA was isolated from plasma using the Qiagen Circulating Nucleic Acids Kit (Qiagen GmbH) and eluted in LoBind tubes (Eppendorf AG). High-molecular weight DNA from white blood cells was extracted using the Qiagen DNA Blood Mini Kit (Qiagen GmbH) followed by shearing using a focused-ultrasonicator (Covaris). Concentration and quality of cfDNA was assessed using the Bioanalyzer 2100 (Agilent Technologies). cfDNA samples with saturated concentrations of high-molecular weight DNA based on fluorescence intensity were excluded from the study.

Next-generation sequencing libraries from cfDNA and sheared high-molecular weight DNA from white blood cells were prepared from 8.4 to 250 ng (Table 2). Genomic libraries were prepared as previously described (Phallen, J. et al. Direct detection of early-stage cancers using circulating tumor DNA. Sci Transl Med 9, doi:10.1126/scitranslmed.aan2415 (2017)). Briefly, the NEBNext DNA Library Prep Kit for Illumina [New England Biolabs (NEB)] was used with four main modifications to the manufacturer's guidelines: i) the library purification steps utilized the on-bead Ampure XP approach, ii) reagent volumes were adjusted accordingly to accommodate the on-bead strategy, iii) a pool of 8 unique Illumina dual index adapters with 8 bp barcodes were used in the ligation reaction, and iv) cfDNA libraries were amplified with HotStart Phusion Polymerase. Genomic library preparation was performed as previously described (Phallen, J. et al. Sci Transl Med 9, doi:10.1126/scitranslmed.aan2415 (2017)). Concentration and quality of cfDNA genomic libraries were assessed using the Bioanalyzer 2100 (Agilent Technologies).

Targeted capture was performed using the Agilent SureSelect reagents and a custom set of hybridization probes targeting 58 genes (Table 3) per the manufacturer's guidelines. The captured library was amplified with HotStart Phusion Polymerase (NEB). The concentration and quality of captured cfDNA libraries was assessed on the Bioanalyzer (Agilent Technologies). Libraries were sequenced using 100-bp paired end runs on the Illumina HiSeq 2500 (Illumina).

Primary processing of next-generation sequencing data and identification of putative somatic mutations using the white blood cell filtering approach: Primary processing of next-generation sequence data for analyses of sequence alterations in cfDNA and white blood cell samples were performed as previously described (Phallen J. et al. 2017). Briefly, Illumina CASAVA (Consensus Assessment of Sequence and Variation) software (version 1.8) was used for demultiplexing and masking of dual index adapter sequences. Sequence reads were aligned against the human reference genome (hg19) using NovoAlign with additional realignment of select regions using the Needleman-Wunsch method (Jones, S. et al. Personalized genomic analyses for cancer mutation discovery and interpretation. Sci Transl Med 7, 283ra253, doi:10.1126/scitranslmed.aaa7161 (2015)).

Candidate tumor-specific mutations in cfDNA, consisting of point mutations, small insertions, and deletions were identified using VariantDx ((Jones, S. et al. 2015) (Personal Genome Diagnostics) across the targeted regions of interest as previously described ((Phallen J. et al. 2017)). Briefly, an alteration was considered a candidate somatic mutation only when: (i) Three distinct paired reads contained the mutation in the cfDNA and the number of distinct paired reads containing a particular mutation in the plasma was at least 0.05% of the total distinct read pairs; or (ii) one distinct paired read contained the mutation in the cfDNA and the mutation had also been detected in at least one additional timepoint at the level specified in (i); (iii) the mismatched base or small indel was not identified in matched white blood cell sequencing data of samples collected at baseline at the level of one distinct read (Table 9); (iv) the mismatched base or small indel was not present in a custom database of common germline variants derived from dbSNP; (v) the altered base did not arise from misplaced genome alignments including paralogous sequences; and (vi) the mutation fell within a protein coding region and was classified as a missense, nonsense, frameshift, or splice site alteration. Candidate alterations were defined as somatic hotspots if the nucleotide change and amino acid change were identical to an alteration observed in ≥20 cancer cases reported in the COSMIC database.

Statistical analyses: Significance was determined using a variety of methods. Wilcoxon rank sum test or Kruskal-Wallis test were performed for continuous variables and Fisher's exact test for categorical variables. Analyses of groups of mutations were carried out in R using the package maftools (Mayakonda, A., et al. Maftools: efficient and comprehensive analysis of somatic variants in cancer. Genome Res 28, 1747-1756, doi:10.1101/gr.239244.118 (2018)). Correlation coefficients were determined for the association between WBC variants and their correspondent alterations identified in cfDNA, as well as for the association between the number of WBC variants and age. Univariate survival analyses and a multivariate Cox proportional-hazards model were carried out in R using packages survival and coxphf (cran.r-project.org).

For mutations identified by cfDNA sequencing but not identified by WBC sequencing, the probability for the model that the mutation was tumor derived was computed relative to the probability for the model that the mutation was hematopoietic. The sampling distribution of the observed number of reads with an altered mutation in cfDNA and WBC sequencing is a binomial parameterized by the total coverage at that mutation and unknown probability theta. Under the tumor derived model, theta_WBC is zero and only theta_plasma is unknown. For the hematopoietic model, it is assumed that theta_WBC and theta_plasma are the same. As a prior for theta_plasma, a beta distribution was used with shape parameters 2.4 and 340 that loosely centers most of the mass on the observed mutation allele frequencies in samples for which mutations were identified in both cfDNA and WBC sequencing. This prior is equivalent to a sample with 2.4 altered reads per 340 distinct molecules. Simulating a large number of theta's from the prior, the probability of the observed data for each simulated theta was computed. The ergodic average of these probabilities approximates the likelihood of the observed data conditional on the model but unconditional on theta. Assuming a prior odds of 1, the posterior odds (PO) was the same as the Bayes factor and the probability that the mutation was tumor derived by PO/(1+PO) was obtained. This analysis was performed for each mutation that was identified only by cfDNA sequencing.

Results

The current study was an exploratory analysis of the predictive value of ctDNA assessment in a subset of patients from CRITICS study (NCT00407186), an investigator-initiated, open-label, multi-center, phase III randomized controlled trial of perioperative chemotherapy (chemotherapy group) versus preoperative chemotherapy with postoperative chemoradiotherapy (chemoradiotherapy group) for patients with resectable gastric cancer (Cats, A. et al. Lancet Oncol 19, 616-628, doi:10.1016/S1470-2045(18)30132-3 (2018)). Between Jan. 11, 2007, and Apr. 17, 2015, a total of 788 patients from 56 hospitals in the Netherlands, Sweden, and Denmark were randomized upfront to receive three preoperative 21-day cycles of intravenous epirubicin, cisplatin or oxaliplatin, and oral capecitabine followed by three postoperative cycles of intravenous epirubicin, cisplatin or oxaliplatin, and oral capecitabine (chemotherapy group) or to receive the same preoperative regimen followed by radiation combined with daily capecitabine and weekly cisplatin (chemoradiotherapy group) (FIG. 1A; FIG. 5; Table 1).

As a proof-of-principle study, matched cfDNA and WBC samples from 50 treatment-naïve patients from the Netherlands who had plasma samples available for genomic analyses at two or more timepoints were sequenced and analyzed to detect tumor-specific mutations in ctDNA (FIG. 1A; FIG. 5; Table 1). The goal was to predict survival outcomes based on ctDNA assessment after preoperative therapy and minimal residual disease analyses after surgery with curative intent. Of the patients analyzed, 24 had diffuse subtype, 24 had intestinal subtype according to Lauren's classification, and one was diagnosed with adenosquamous gastric carcinoma (FIG. 1B; Table 1). Twenty-one patients were surgically treated with distal gastrectomy, 17 with total gastrectomy, four with esophagocardiac resection, and one with proximal gastrectomy (FIG. 1B; Table 1). Despite initial eligibility at the time of treatment enrollment, six patients showed evidence of advanced disease during the exploratory laparotomy and were not submitted to surgical resection. One additional patient did not undergo surgical treatment for unknown reasons. Histopathological regression after preoperative therapy was determined according to Mandard's tumor regression grade (TRG). Three patients achieved complete regression after three cycles of preoperative chemotherapy at the time of surgery (TRG 1) while 10, 15, 13, and 2 patients presented with pathological stage I, II, III, and IV, respectively (FIG. 1B; Table 1). Centrally reviewed pathological assessment of resection specimens after three cycles of preoperative chemotherapy showed that 20 patients did not have evidence of lymph node involvement (ypN0), while 23 patients had lymph node infiltration, including 10 patients with ypN1 (including 3 ypN1mi), 7 patients with ypN2, and 6 patients with ypN3 disease (Table 1). Twenty-six patients received postoperative treatment with radiation combined with cisplatin and capecitabine, and 24 patients were postoperatively treated with three cycles of epirubicin, cisplatin, and capecitabine without radiation after surgery (FIG. 1B; Table 1).

For each patient, plasma and buffy coat were collected at the time of trial enrollment (baseline timepoint), after patients received three cycles of preoperative chemotherapy (preoperative timepoint), and after surgery but before the initiation of the adjuvant treatment (postoperative timepoint) (FIG. 1A; Tables 1 and 2). An approach was developed to identify tumor-specific alterations in the circulation independent of tissue analyses by parallel deep sequencing of cfDNA and WBCs, followed by identification of cfDNA alterations and removal of hematopoietic-related changes detected in WBCs (FIG. 1A). For sequencing analyses of cfDNA and WBCs, a next generation deep sequencing approach was used to evaluate 58 cancer driver genes (FIG. 1A; Tables 3, 4, and 5). This method is based on targeted capture and deep sequencing (>30,000×) of DNA fragments to identify single base substitutions and small insertions or deletions in cfDNA across 80,930 bp of coding gene regions while distinguishing these from PCR amplification and sequencing artifacts (Phallen, J. et al. Sci Transl Med 9, doi:10.1126/scitranslmed.aan2415 (2017)). For alterations detected in cfDNA that were not identified by matched white blood cell sequencing, the posterior probability that such an alteration was tumor derived from a Bayesian statistical model was determined using the frequency of altered alleles and total coverage of cfDNA and WBCs sequences.

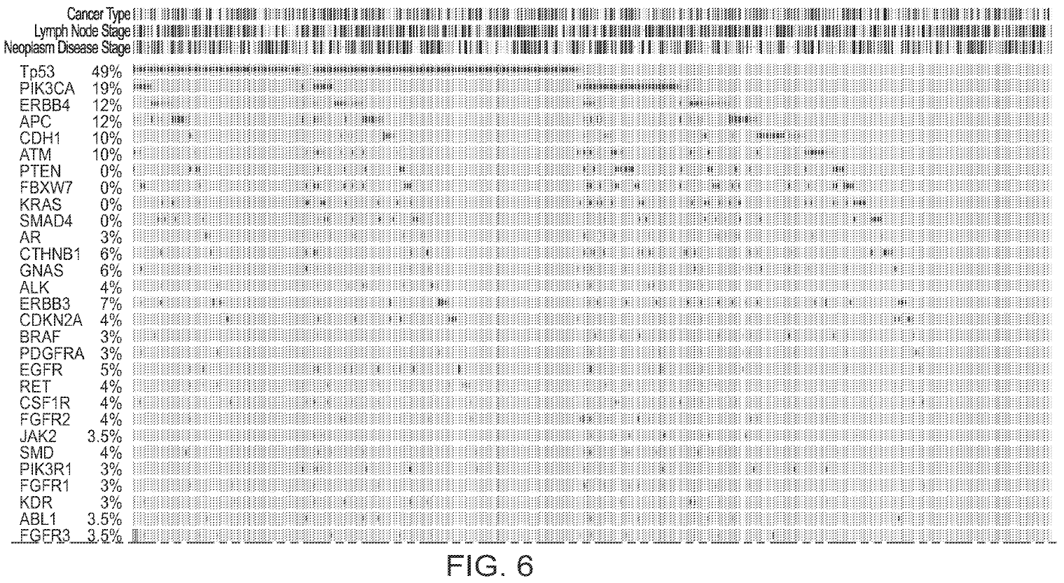

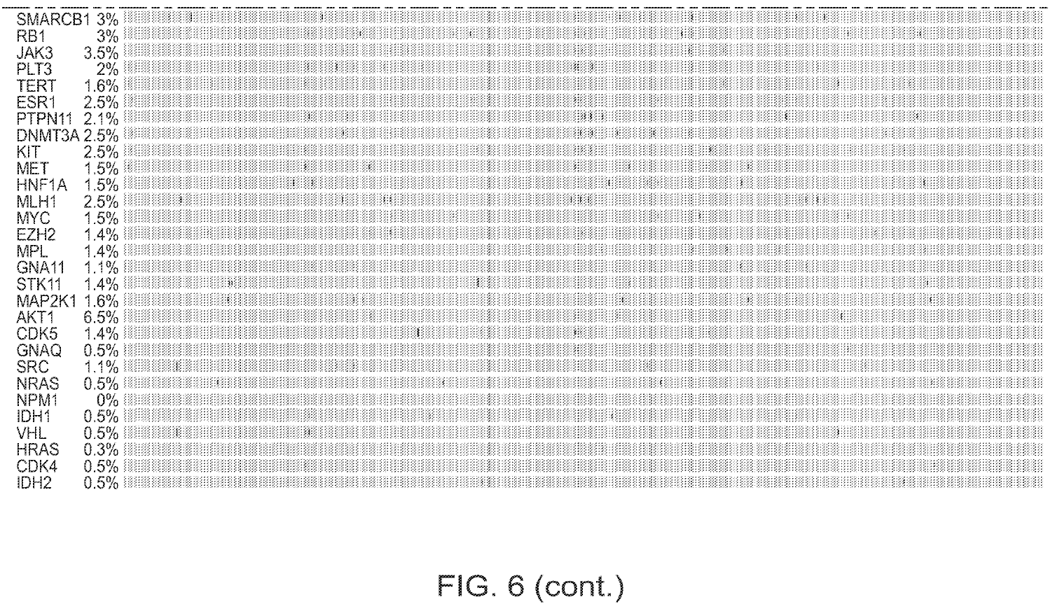

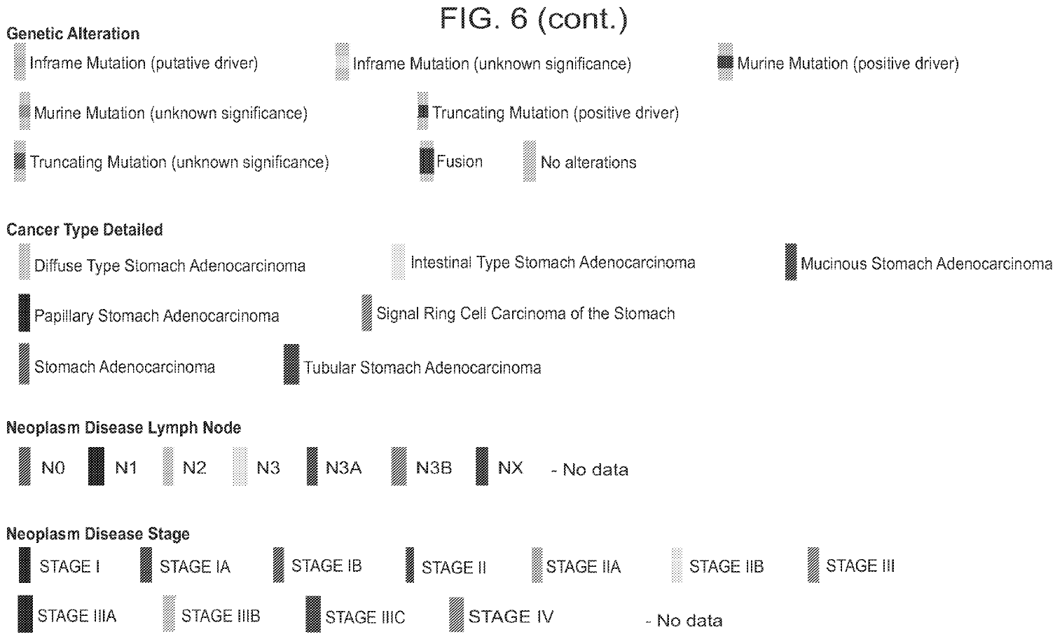

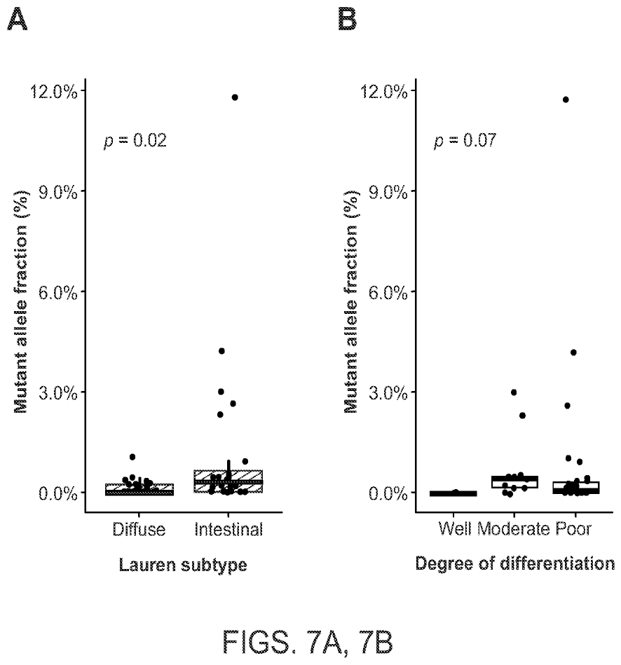

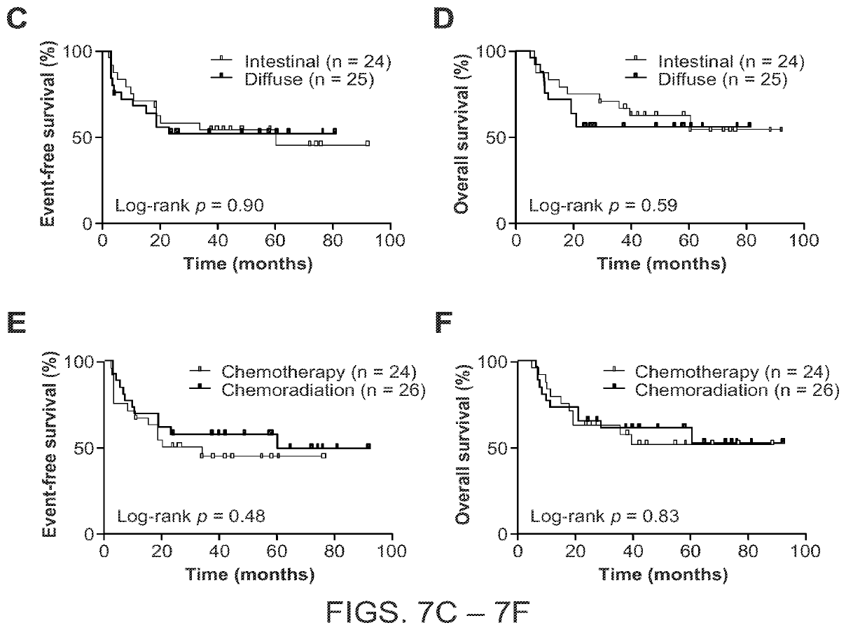

To estimate the theoretical sensitivity of detection of the sequencing approach in gastric cancer, the proportion of gastric adenocarcinomas in the TCGA Pan-Cancer Atlas (Hoadley, K. A. et al. Cell-of-Origin Patterns Dominate the Molecular Classification of 10,000 Tumors from 33 Types of Cancer. Cell 173, 291-304 e296, doi:10.1016/j.cell.2018.03.022 (2018)) with alterations in one or more of the 58 analyzed genes was determined. These analyses showed that our targeted panel would have a sensitivity of ˜88% as 384 of 436 gastric cancer cases had at least one alteration in these genes (FIG. 6). Overall, median levels of mutant allele fractions at baseline were observed to be significantly higher in patients with intestinal subtype when compared to diffuse subtype (0.295% vs 0%, p=0.015, Wilcoxon rank sum test) (FIG. 7A). There was no statistically significant difference among levels of mutant allele fractions among well, moderately, or poorly differentiated tumors (p=0.07, Kruskal-Wallis test) (FIG. 7B). In this study, patients with intestinal and diffuse gastric adenocarcinoma experienced similar event-free (Table 1; FIG. 7C) and overall survival (Table 1; FIG. 7D). Consistent with the findings of the original trial (Cats, A. et al. Lancet Oncol 19, 616-628, doi:10.1016/S1470-2045(18)30132-3 (2018), significant differences were not observed in survival outcomes related to the postoperative treatment arm in which patients had been randomized (Table 1; FIGS. 7E and 7F).

Detection of clonal hematopoiesis and identification of tumor-specific alterations: cfDNA was evaluated in all 50 patients at baseline and after 3 cycles of preoperative chemotherapy. At baseline, sequence alterations were detected in cfDNA from 40 patients (80%) (FIG. 2A; Table 6) and in WBCs from 31 patients (62%) (FIG. 2B; Table 7). After removing WBC-derived alterations from cfDNA data, 54 alterations were detected that were likely tumor-specific in 27 patients (54%) (FIG. 2C; Table 8). The most frequently altered genes detected in WBCs were DNMT3A (45%), TP53 (29%), EGFR (10%), APC (6%), AR (6%), ATM (6%), and MLH1 (6%), while the most frequently altered genes detected in ctDNA were TP53 (22%), MYC (15%), PIK3CA (15%), KRAS (11%), HRAS (11%), BRAF (11%), ALK (11%), ATM (11%), KIT (11%), and CDH1 (7%) (FIGS. 2B and 2C; Table 8). In accord with the molecular classification of gastric adenocarcinomas proposed by the TCGA (Cancer Genome Atlas Research, N. Comprehensive molecular characterization of gastric adenocarcinoma. Nature 513, 202-209, doi:10.1038/nature13480 (2014)), a higher frequency (60%) of PIK3CA mutations was found in the blood of patients with EBV positive (n=3) or MSI-high tumors (n=2) compared to the frequency (3%) in EBV negative (n=36) and MSS tumors (n=37). The median mutant allele fraction among 43 WBC-derived variants was 0.31% (IQR 0.18%-0.63%), which was similar to the median mutant allele fraction among 53 tumor-specific variants identified in cf-DNA (0.31%, IQR 0.20%-0.55%, p=0.96, Wilcoxon rank sum test) (FIG. 2D). A high correlation was observed between levels of mutant allele fractions in WBCs and levels of corresponding alterations in cfDNA (Pearson correlation coefficient=0.91) (FIG. 2E). The number of alterations detected in WBCs increased with age (r²=0.36, exponential correlation) (FIG. 2F).

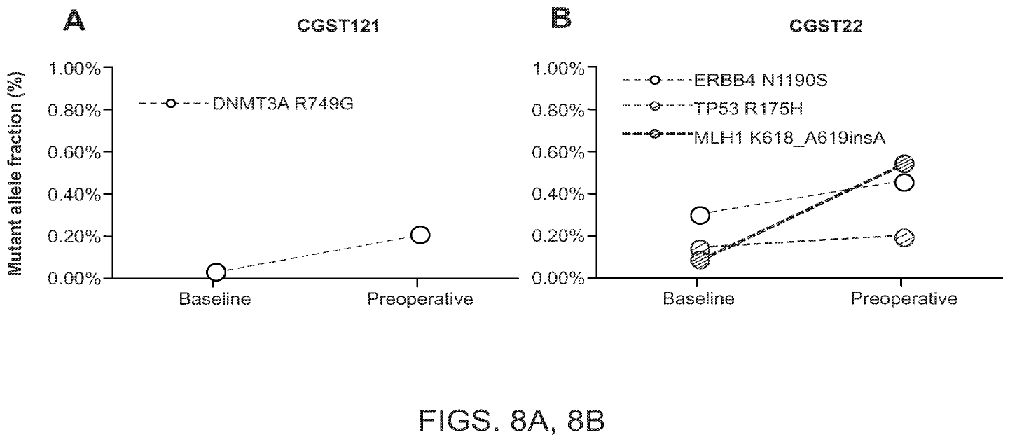

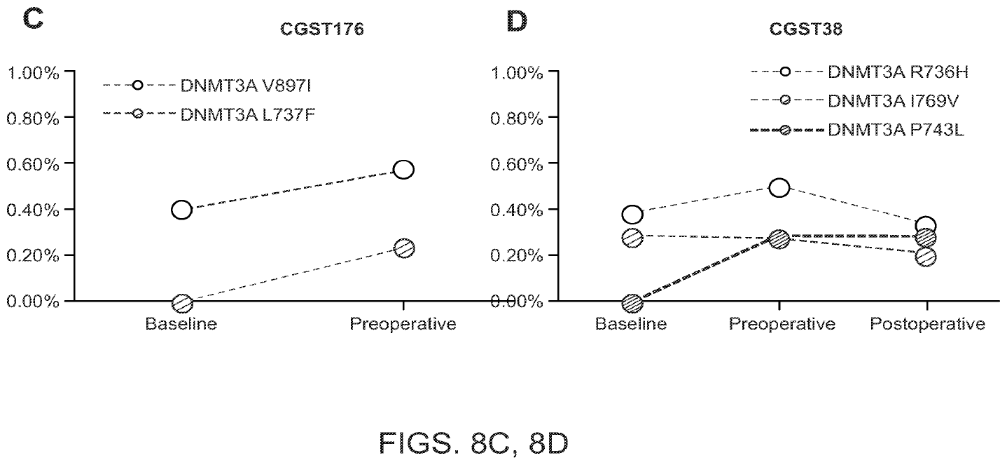

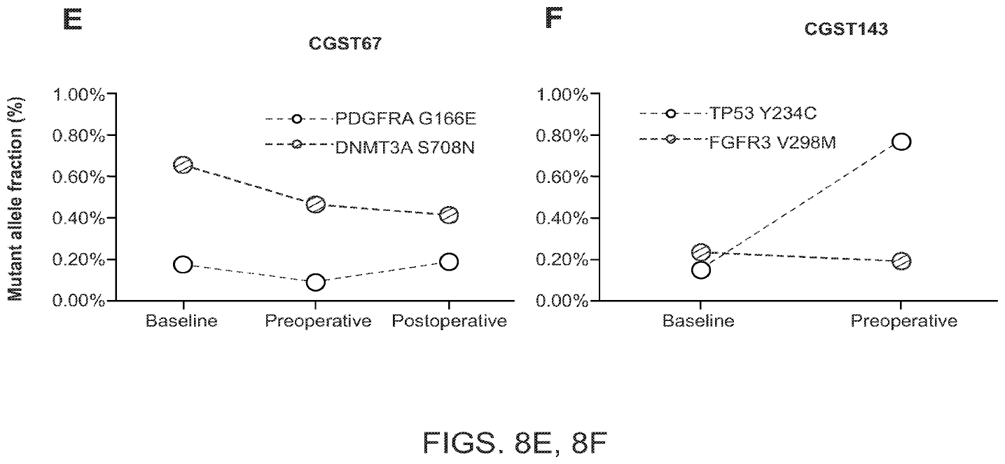

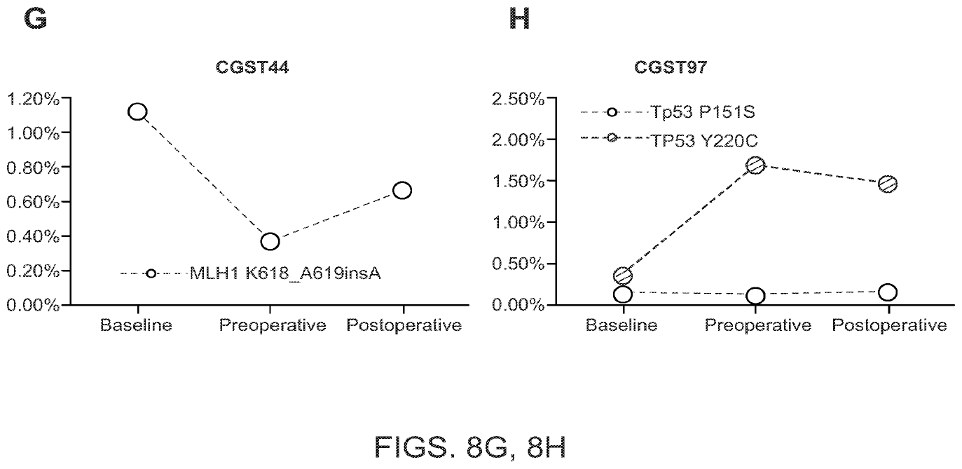

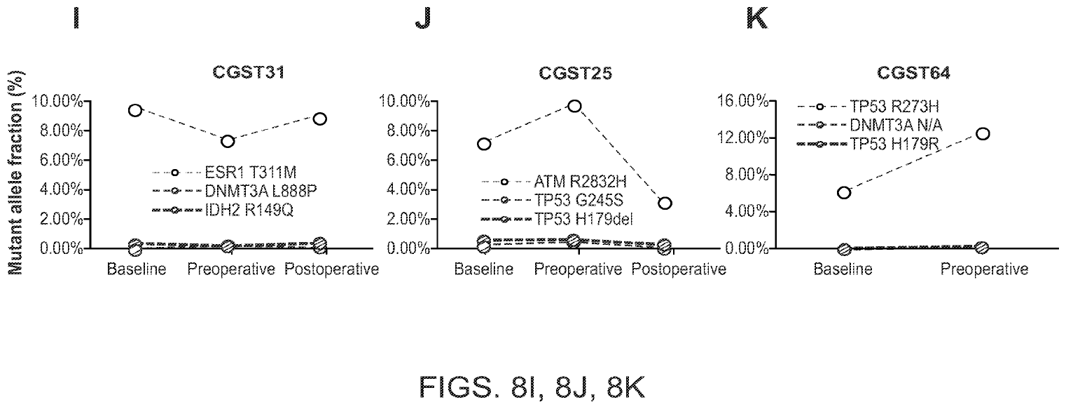

Twenty one sequence alterations were observed in TP53 in cfDNA, including 17 missense mutations, two nonsense mutations, one in-frame deletion, and one splice site mutation (FIG. 2G; Table 6). Of the cfDNA sequence changes observed in TP53, 15 were identified in WBC sequences as well as three alterations in WBC's that were not present in matched cf-DNA (FIG. 2G; Table 7). From the 21 TP53 alterations initially detected in cfDNA at baseline, only 6 were identified as tumor-specific mutations, including two with stop alterations (Q192* and S166*) as well as four missense mutations (R175H, V216M, N239S, R248W). Fragment length distributions of the 21 TP53 alterations detected in cfDNA were further evaluated. It was observed that fragments harboring tumor-specific TP53 mutations in the circulation were significantly shorter than fragments harboring TP53 variants associated with clonal hematopoiesis (p<0.001, Kolmogorov-Smirnov test), as well as fragments harboring wild-type TP53 coding regions (p<0.001, Kolmogorov-Smirnov test) (FIG. 2H; Tables 7, 8, and 9). Interestingly, WBC variants were detected in DNMT3A, TP53, ERBB4, MLH1, PDGFRA, FGFR3, ESR1, IDH2, and ATM among multiple time points analyzed in 11 patients that did not harbor any tumor-specific alterations in cfDNA (FIGS. 8A-8K). Overall, detection of WBC variants or tumor-derived ctDNA variants at baseline did not reveal statistically significant differences in event-free or overall survival (FIGS. 9A-9D).

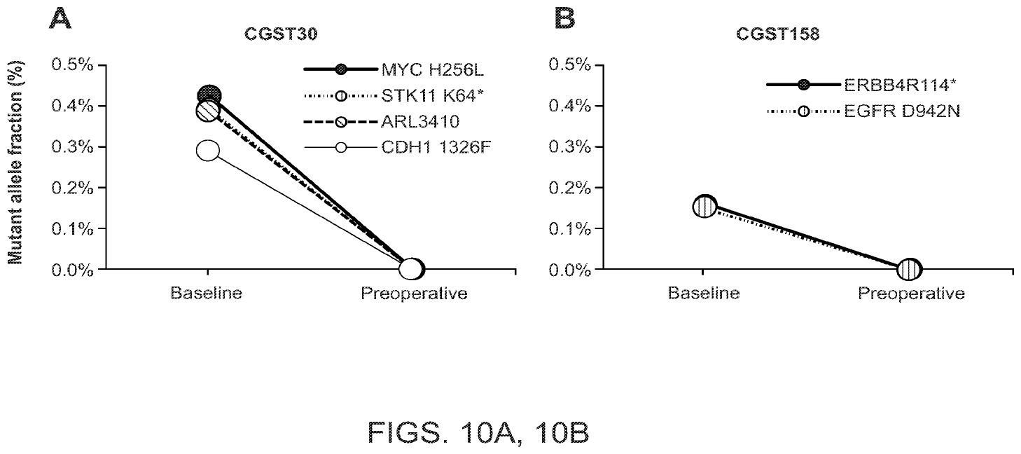

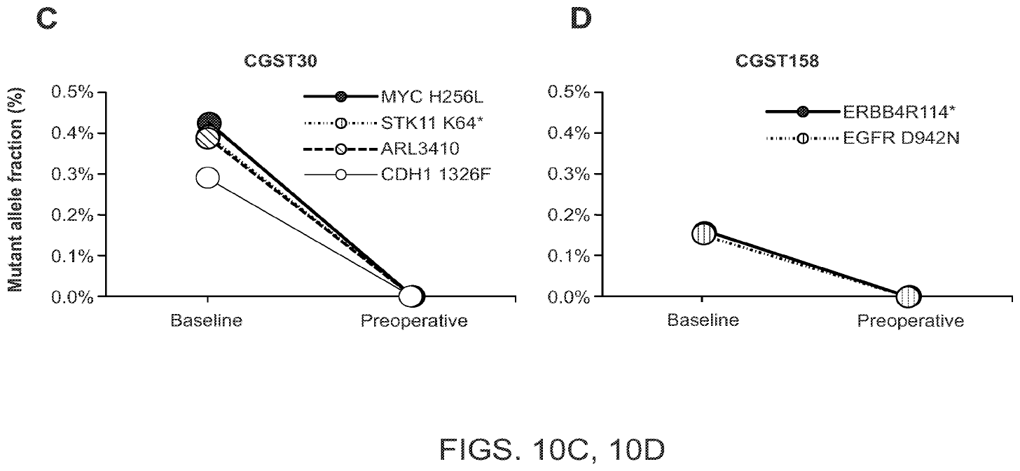

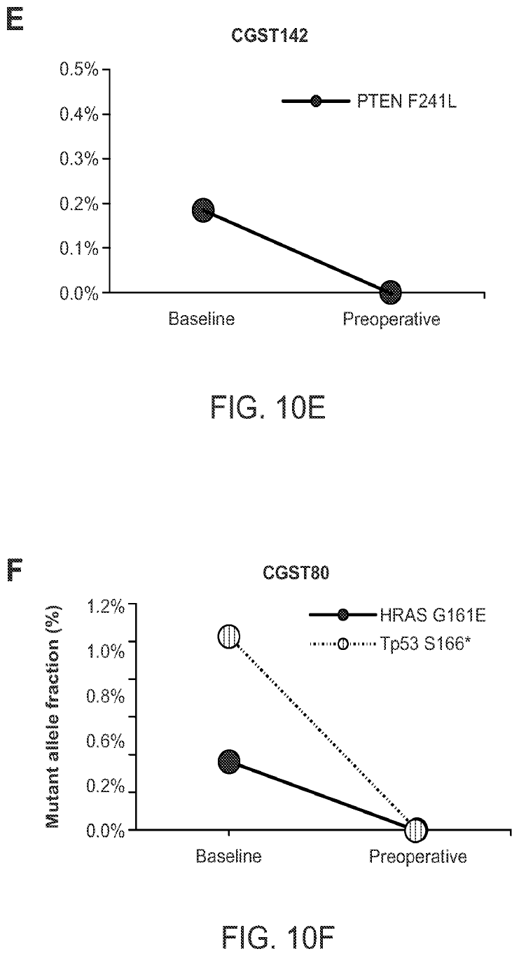

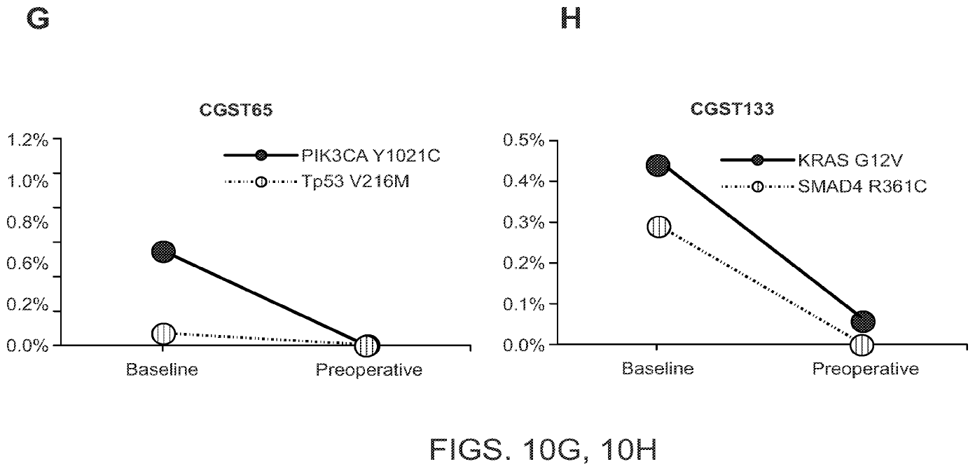

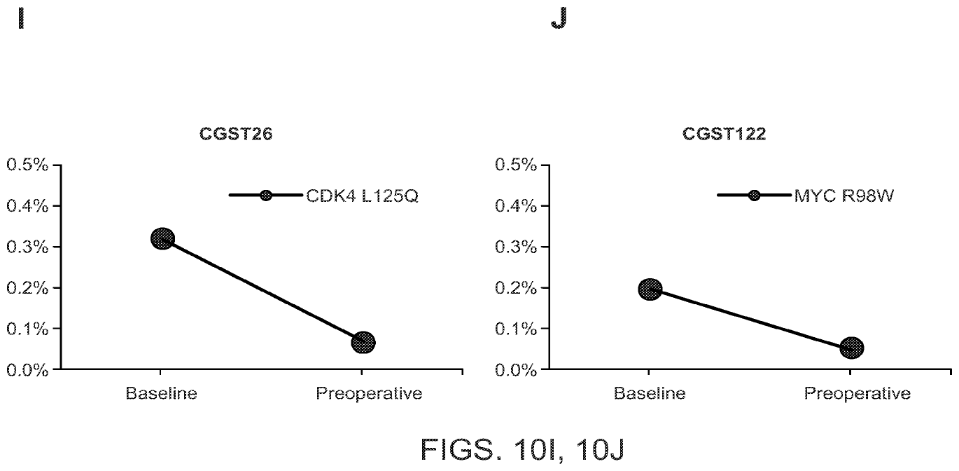

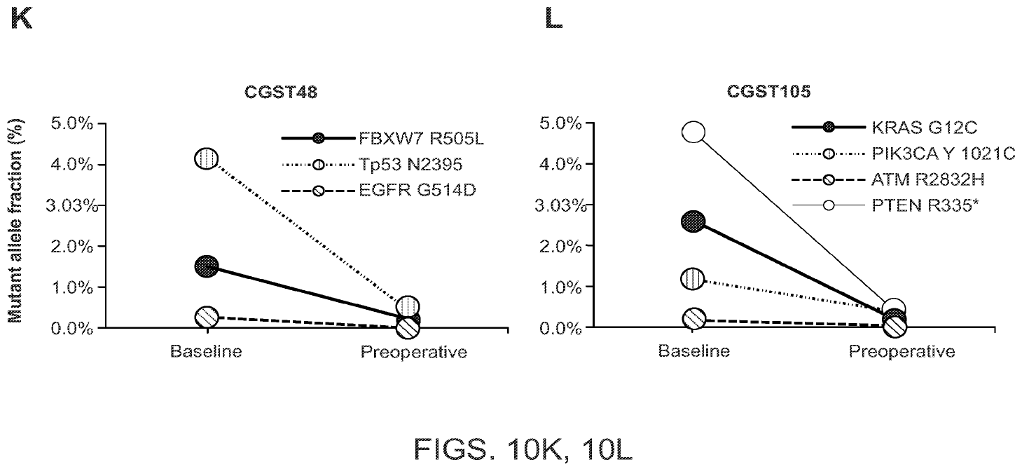

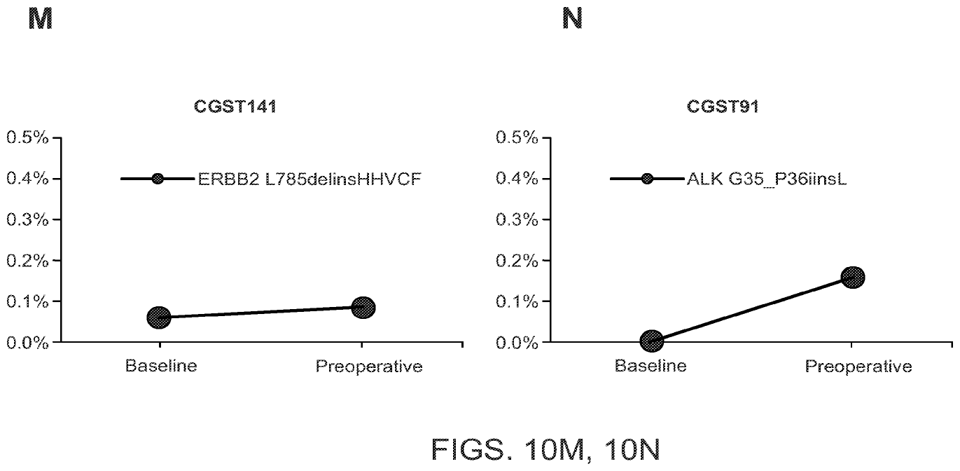

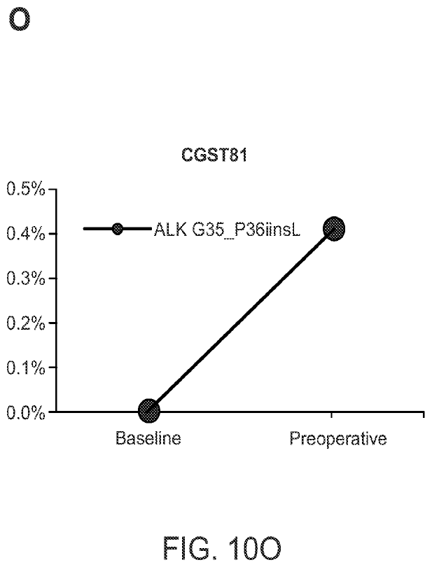

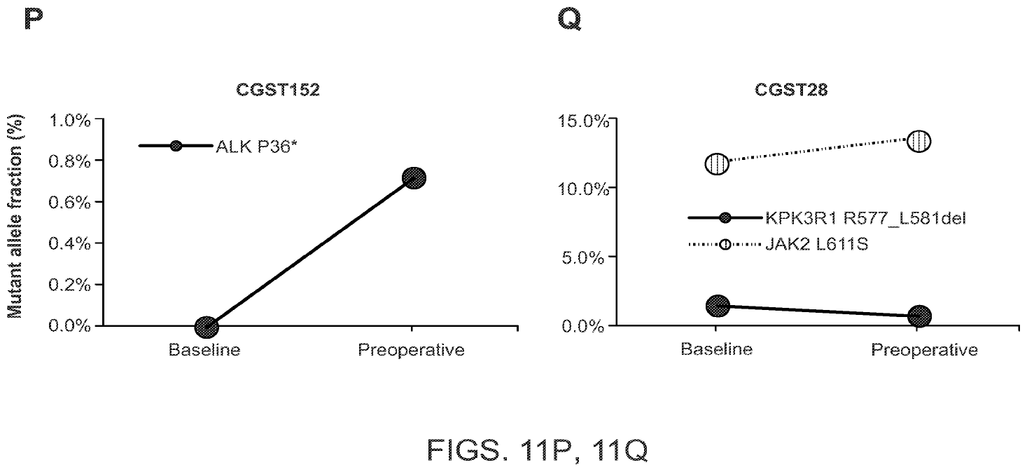

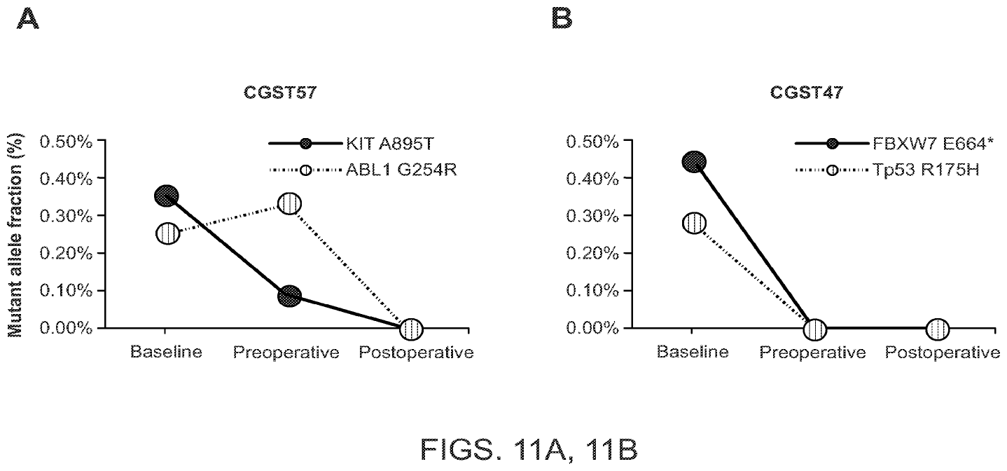

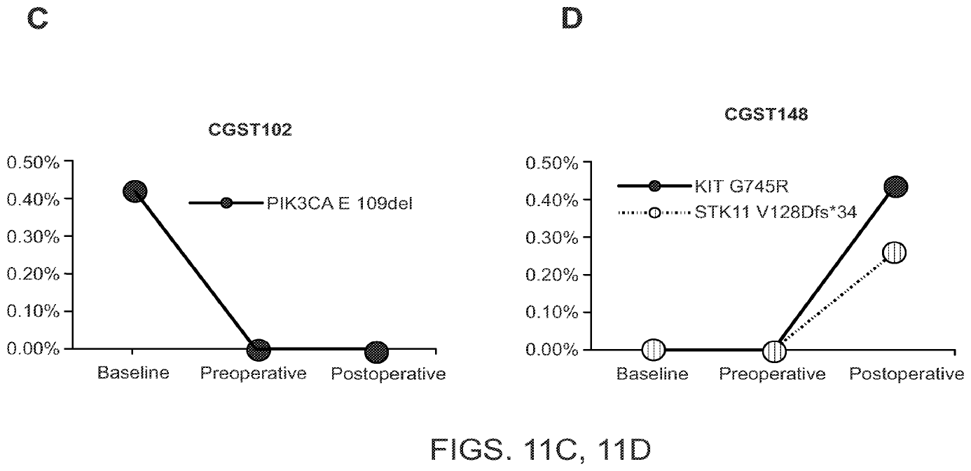

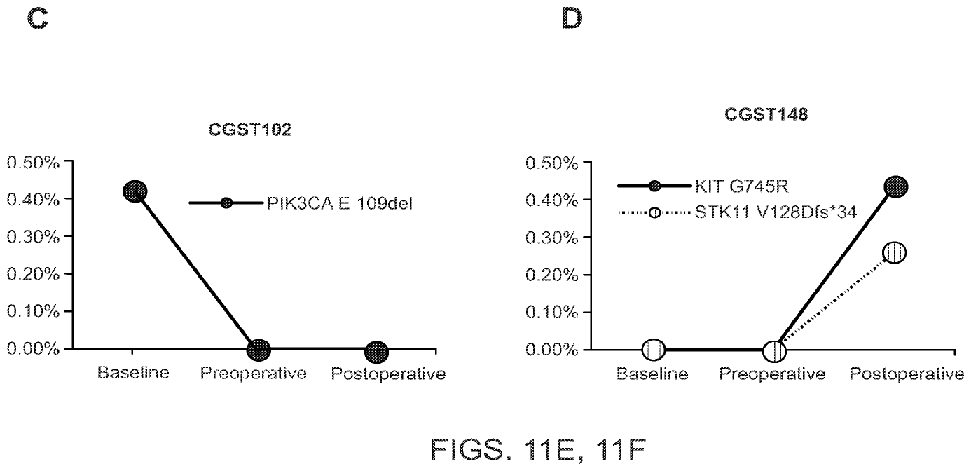

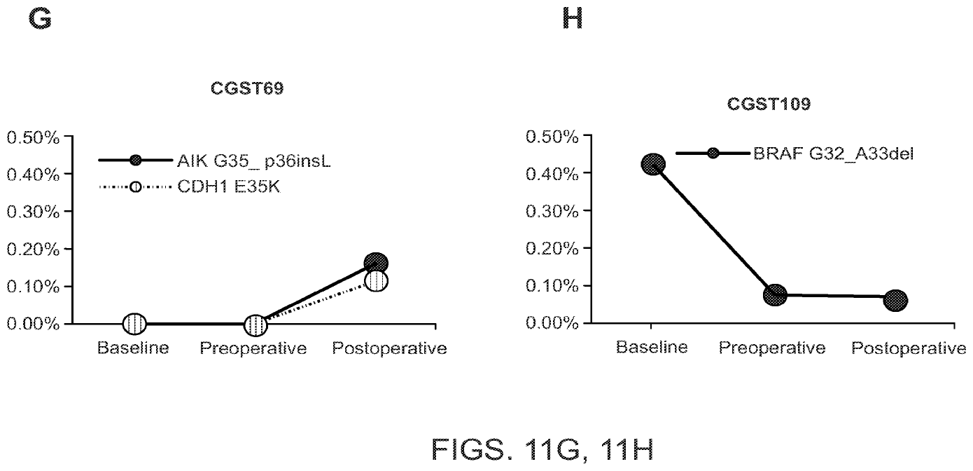

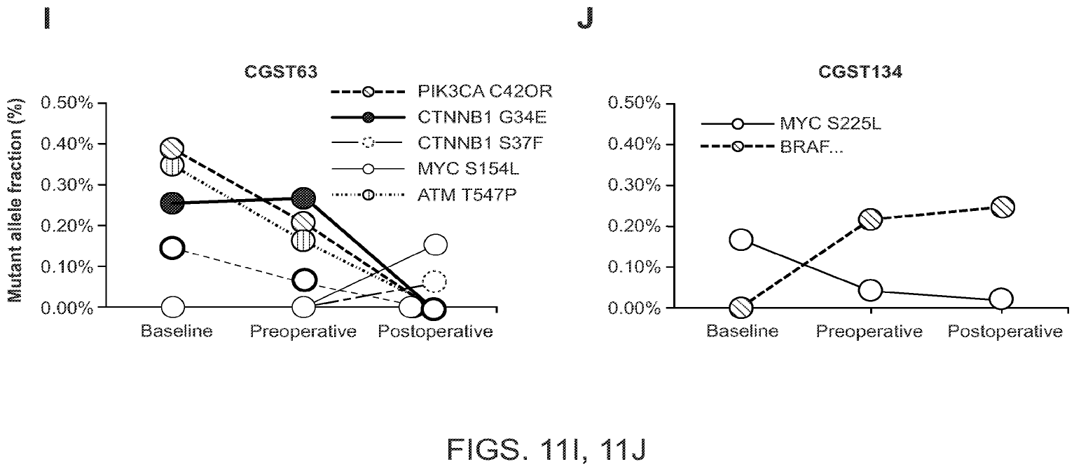

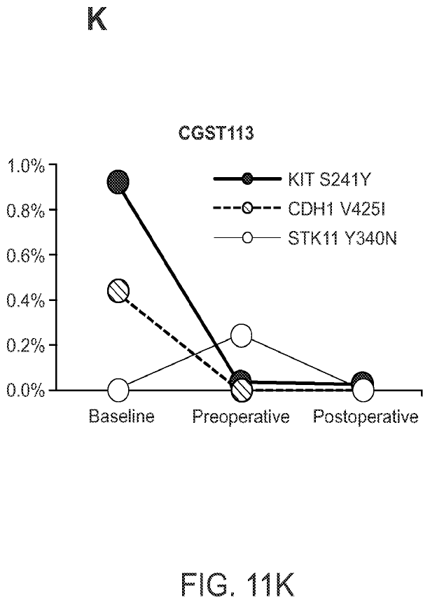

Preoperative ctDNA is a surrogate biomarker for pathological response in gastric cancer: After identification of ctDNA alterations using the parallel sequencing of cfDNA and WBCs indicated above, ctDNA levels were evaluated before and after preoperative chemotherapy. Of the 30 patients with measurable ctDNA at baseline or at the preoperative time point after filtering WBC sequence alterations (FIG. 2C), 11 experienced a complete elimination of ctDNA levels after nine weeks of systemic treatment (FIGS. 10A-10Q and 11A-11K; Table 5). As an example, patient CGST33, who presented with intestinal subtype gastric adenocarcinoma at diagnosis had mutant allele fraction concentrations of 2.32% and 0.64% for TP53 Q192* and ERBB2 R756Cfs*2, respectively that were completely eliminated at the preoperative timepoint. This drop in ctDNA occurred in conjunction with a major pathological response (TRG 2) in the specimen obtained at the time of surgery (FIG. 3A). In contrast, 19 patients had detectable ctDNA at the preoperative timepoint (FIGS. 10A-10Q and 11A-11K; Table 5), including as an example in patient CGST110 who had mutant allele fractions of 0.15% for ERBB4 T639M at baseline and 0.12% at the preoperative timepoint (FIG. 3B). This patient did not experience tumor regression after nine weeks of systemic treatment (TRG 5) and eventually died from recurrent disease 35 months after the initial diagnosis (Table 1).

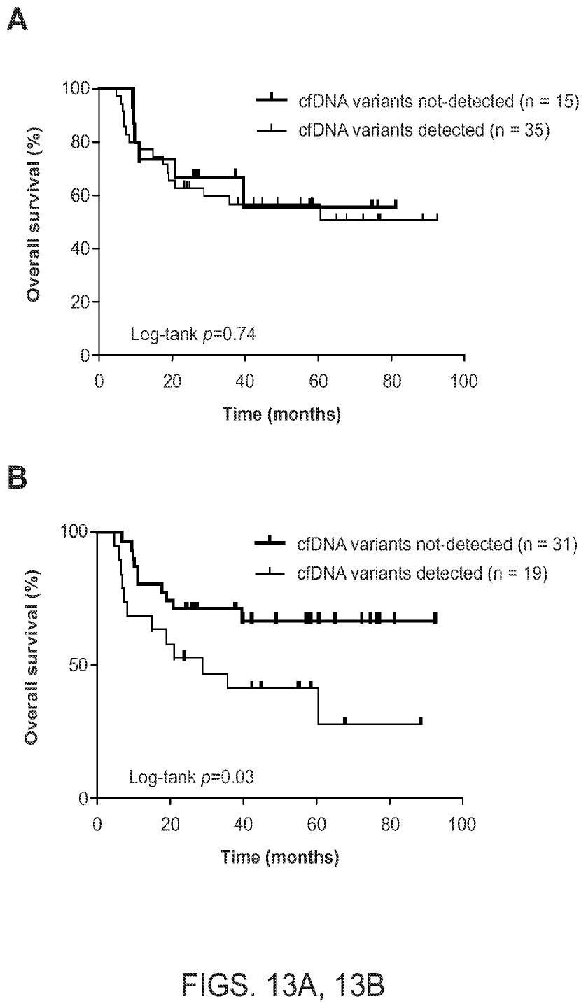

After preoperative chemotherapy, seven responders were identified, of whom three achieved complete pathological response (TRG 1) and four achieved a major pathological response, exhibiting fibrotic surgical specimens with scattered tumor cells (TRG 2). All seven responders had no ctDNA detected at the preoperative timepoint (FIG. 3C). Patients with lower degrees of tumor regression (TRG 3-5) and at least one involved lymph node (ypN1, ypN2, and ypN3) presented more frequently with detectable ctDNA at the preoperative timepoint (FIG. 3C; FIG. 12A). In contrast, the absence of ctDNA at the preoperative timepoint was significantly associated with major pathological regression (TRG 1-2) at the time of surgery (p=0.03, Fisher's exact test) (FIG. 3D). As expected, recurrence was also associated with lower degrees of pathological response (p=0.03, Fisher's exact test) (FIG. 3E), at least one involved lymph node (p=0.002, Fisher's exact test) (FIG. 3F), and detectable ctDNA at the preoperative timepoint (p=0.02, Fisher's exact test) (FIG. 3G). TRG score (TRG 1-2 versus TRG 3-5) and pathological lymph node status (ypN0 versus ypN+) were strongly associated with survival outcomes (FIGS. 12B and 12C). It is noteworthy that detection of mutations in cfDNA without a WBC sequence filter at the preoperative timepoint did not predict risk of recurrence (FIG. 3H) or death (FIG. 13A). However, when the WBC-guided hematopoietic filter was applied, it was observed that ctDNA detected at the preoperative timepoint was associated with a significantly higher risk of recurrence and shorter median event-free survival (18.4 months versus median not reached) (Log-rankp=0.012; HR=3.0; 95% CI=1.3-6.9) (FIG. 3I) as well as higher risk of death and shorter median overall survival (28.7 months versus median not reached) (Log-rankp=0.03; HR=2.7; 95% CI=1.1-6.7) (FIG. 13B).

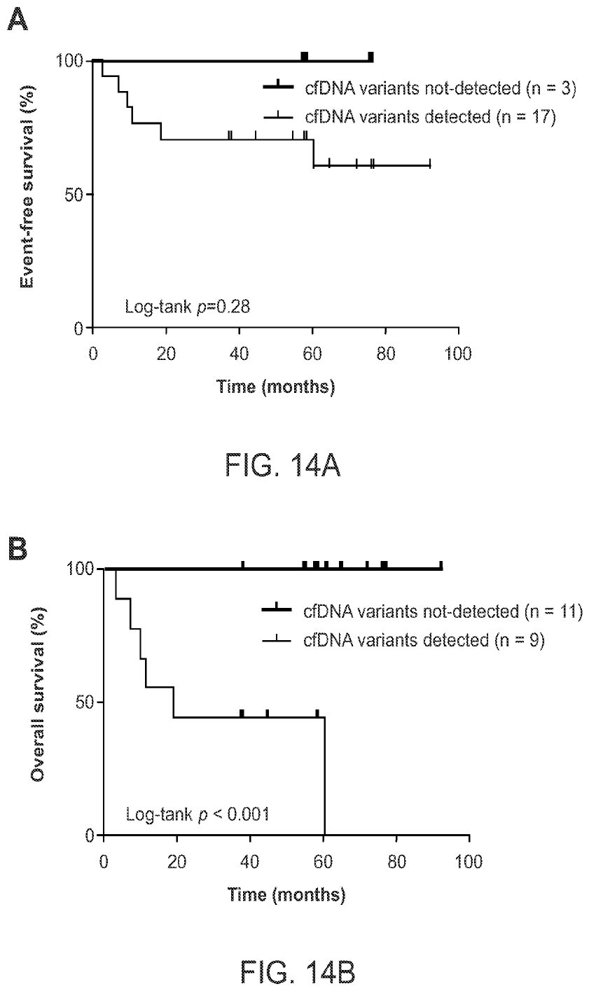

Minimal residual disease predicts survival outcome after surgery in gastric cancer: The WBC-filtering approach was used to evaluate minimal residual disease after surgery from all 20 patients with blood samples available from a postoperative timepoint. Blood samples were collected at a median time of 6.5 weeks after surgery (Table 1). Complete elimination of tumor-specific mutations in cfDNA was observed at the postoperative time point for four patients with major tumor responses (TRG 1 and TRG 2), including in patient CGST32, who exhibited baseline mutant allele fraction concentrations of 0.65% and 0.24% for BRAF G469A and KRAS G13R, respectively (FIG. 4A). The two hotspot ctDNA mutations in this patient were not detected at either the pre- or postoperative timepoint, in agreement with the surgical specimen assessment that showed major tumor regression (TRG 2) (FIG. 4A; Tables 1 and 8). In contrast, postoperative tumor specific mutations were detected in nine out of 16 patients with minor or no pathologic tumor responses (TRG 3-5), including in patient CGST68, who presented with mutant allele fraction of 0.03% for HRAS D54Efs*53 frameshift mutation at the baseline timepoint. This patient exhibited progressive increases in mutant allele fractions of HRAS at preoperative and postoperative timepoints, followed by the emergence of ERBB4 D 1184*, detected at 0.16% mutant allele fraction after surgery (FIG. 4B; Tables 1 and 8). After a median follow-up of 42 months, it was observed that all eleven patients without detectable tumor-specific mutations at the postoperative timepoint were alive and free of recurrence (FIG. 4C; Table 1). On the other hand, six out of nine patients with detectable tumor-specific mutations at the postoperative timepoint developed disease recurrence and died from metastatic disease (FIG. 4C; Table 1). Again, detection of mutations in cfDNA without a WBC filter after surgery did not predict recurrence (FIG. 14A) or death (FIG. 4D). In contrast, with the WBC-guided hematopoietic filter, a significant shorter median event-free survival and a higher risk of disease recurrence was observed for patients with detectable tumor-specific mutations after surgery (18.7 months versus median not reached; Log-rank p<0.001; HR=21.8; 95% CI=3.9-123.1) (FIG. 14B) as well as a significantly shorter median overall survival (28.7 versus median not reached; Log-rank p<0.001; HR=21.8; 95% CI=3.9-123.1) (FIG. 4E).

Discussion

High mortality rates associated with gastric cancer reflect the prevalence of advanced disease at presentation, when treatment options are limited (Van Cutsem, E., et al. Lancet 388, 2654-2664, doi:10.1016/S0140-6736(16)30354-3 (2016)). Despite the value of multimodal curative treatment approaches, a significant fraction of patients will eventually perish as a consequence of locoregional relapse, peritoneal recurrence, or distant metastases (Songun, I., et al. Lancet Oncol 11, 439-449, doi:10.1016/S1470-2045(10)70070-X (2010). Bickenbach, K. A., et al. Ann Surg Oncol 20, 2663-2668, doi:10.1245/s10434-013-2950-5 (2013)). Current methods to estimate the risk of disease recurrence after surgery mostly rely on the assessment of pathological staging and microscopic residual disease score systems (Becker, K. et al. Cancer 98, 1521-1530, doi:10.1002/cncr.11660 (2003). Smyth, E. C. et al. J Clin Oncol 34, 2721-2727, doi:10.1200/JCO.2015.65.7692 (2016). Langer, R. & Becker, K. Virchows Archiv: an international journal of pathology 472, 175-186, doi:10.1007/s00428-017-2232-x (2018)). However, there are several limitations with these approaches, especially with tumor regression grading scales, that make their implementation difficult in daily clinical practice, including interobserver variability and lack of standardization. Furthermore, the poor sensitivity of currently available imaging methods and blood protein biomarkers to detect remaining disease after curative surgery has provided an opportunity for ctDNA analyses for minimal residual disease assessment in gastric cancer. Here, a tissue-independent sequencing approach was developed using ultrasensitive sequencing of matched cfDNA and white blood cells to detect tumor-specific mutations in cfDNA after completion of preoperative chemotherapy as well as after surgery in patients with resectable gastric cancer.

Current evidence-based perioperative strategies for gastric cancer with curative intention encompass perioperative chemotherapy, postoperative chemoradiotherapy and postoperative chemotherapy (Cunningham, D. et al. N Engl J Med 355, 11-20, doi:10.1056/NEJMoa055531 (2006). Macdonald, J. S. et al. N Engl J Med 345, 725-730, doi:10.1056/NEJMoa010187 (2001). Sasako, M. et al. J Clin Oncol 29, 4387-4393, doi:10.1200/JCO.2011.36.5908 (2011)). However, these treatment approaches suffer from poor patient compliance, particularly after surgical resection. In the recently published phase III clinical trials investigating perioperative strategies for resectable gastric cancer only 50-60% of patients could complete the postoperative treatment regimens due to toxicity, disease progression or refusal (Cats, A. et al. Lancet Oncol 19, 616-628, doi:10.1016/S1470-2045(18)30132-3 (2018). Al-Batran, S. E. et al. Lancet 393, 1948-1957, doi:10.1016/S0140-6736(18)32557-1 (2019)). Benefit from perioperative treatment is now thought to be derived from the preoperative part of the treatment. The currently conducted CRITICS-II trial therefore focuses on neoadjuvant strategies and does not include any adjuvant treatment (Slagter, A. E. et al. BMC Cancer 18, 877, doi:10.1186/s12885-018-4770-2 (2018)). There is however still an urgent clinical need to select patients who do need adjuvant treatment because of the presence of minimal residual disease. Here a new ctDNA approach for detection of MRD that could select patients for adjuvant strategies is presented herein. The findings herein, support the investigation of real-time minimal residual disease assessment based on ctDNA analyses after surgery in future interventional trials to address the clinical utility of such an approach to assist clinicians in the decision-making process of selecting patients in need of adjuvant treatment.

The study herein is the first study to investigate the value of parallel deep sequencing of cfDNA and WBCs to detect cfDNA alterations associated with clonal hematopoiesis in the circulation and to use this approach to longitudinally identify bonafide tumor-specific alterations. This approach allows direct identification of ctDNA without requiring tumor tissue, which is often available to a limited extent and where sequencing analyses may be hampered by intra-tumoral heterogeneity. It was also demonstrated herein, that plasma samples from patients with Lauren's intestinal subtype were associated with higher mutant allele fractions when compared with patients with diffuse subtype tumors.

A major challenge for the development of MRD assays using noninvasive liquid biopsies is distinguishing tumor-specific mutations from background changes associated with biological variation. The vast majority of cfDNA in healthy individuals arises from hematopoietic cells (Moss, J. et al. Nat Commun 9, 5068, doi:10.1038/s41467-018-07466-6 (2018)). Normal ageing is associated with the accumulation of somatic mutations in bone marrow-derived hematopoietic cells in the form of CHIP in asymptomatic individuals (Xie, M. et al. Nat Med 20, 1472-1478, doi:10.1038/nm.3733 (2014)). WBC-derived alterations that arise as a consequence of CHIP may confound liquid biopsy analyses that are based on characterization of cfDNA as these may occur in common cancer driver genes, as observed with hotspot alterations in TP53 and KRAS (Hu, Y. et al. Clin Cancer Res 24, 4437-4443, doi:10.1158/1078-0432.CCR-18-0143 (2018)). As shown in the cohort of 50 patients, cfDNA analyses without WBC filters would have been unable to appropriately identify patients that benefit from perioperative treatment in terms of event-free and overall survival.

It was reported herein, that a tissue-independent approach designed to detect tumor-specific cfDNA alterations in patients with resectable gastric cancer treated with perioperative chemotherapy can be applied to predict treatment response and identify patients under higher risk of disease relapse. Recent ctDNA analyses to assess response to preoperative immune checkpoint blockade for patients with stage III non-small cell lung cancer similarly revealed dramatic molecular responses in the circulation in individuals with major pathological responses (Anagnostou, V. et al. Cancer Res 79, 1214-1225, doi:10.1158/0008-5472.CAN-18-1127 (2019)). These results reinforce a paradigm of using ctDNA analyses for response to therapy and minimal residual disease assessment in solid tumors. The approach herein provides evidence that noninvasive detection of ctDNA would be useful for early risk stratification of patients with gastric cancer and therapeutic decisions for novel interventions in clinical trials.

### Example 2: Early Detection and Detection of Minimal Residual Disease in Stage II and III Colorectal Cancer Patients Using a Noninvasive, White Blood Cell-Guided Liquid Biopsy Approach to Identify Mutations as Biomarkers

Over 1.2 million individuals are diagnosed with colorectal cancer (CRC) every year and more than 608,000 deaths occur annually making it the third most common cancer as well as the third highest cause of cancer related death in the developed world (1, 2). CRC can be curable at early stage when the tumor is detected and removed however the disease often develops without symptoms until advanced stage (3, 4). High morbidity and mortality are associated with diagnoses at late stages when less effective surgical and therapeutic interventions are available. There is an urgent need to develop effective screening and early detection strategies to move the detection of disease from late to early stage. Currently, there is a lack of effective biomarkers for CRC screening and interception. Colonoscopy is a useful means of identifying CRC, however the procedure is invasive, requires skilled practitioners, places a burden on healthcare systems in terms of cost and workforce needs, and has suboptimal compliance with only ˜64% of the United States population participating in regular screening (5). CEA is a biomarker of recurrence but is not useful for screening (6, 7), and other possible noninvasive strategies such as fecal occult blood testing or methylated SEPT9 testing suffer from low compliance and specificity (8-11). Detection of minimal residual disease post-resection in early stage colorectal cancer patients could be improved beyond the current standard of care. Patients diagnosed with stage II CRC have a surgical resection but no additional therapy; 20% of patients recur within 5 years indicating that these patients may benefit from additional treatment (12-18). Stage III patients with CRC undergo surgical resection and adjuvant chemotherapy, however a subset of these individuals may be cured with surgery alone.

Development of noninvasive liquid biopsy methods based on analyses of cell-free DNA (cfDNA) provides the opportunity for early detection and detection of minimal residual disease post resection in CRC patients through sensitive and specific direct detection of circulating tumor DNA (ctDNA). Next-generation sequencing technologies together with advanced bioinformatics have brought ctDNA-based assays to the forefront of genotyping in a variety of cancer types, however current studies have mostly been applied to patients with late-stage cancers or have used tumor tissue sequencing to guide mutational analyses in the blood (19-26). Screening and early detection of cancer require direct detection of ctDNA in the blood with no prior knowledge of tumor presence and high specificity yet existing approaches have suffered from a background of hematopoietic alterations identified in the plasma. We here present a white blood cell-guided liquid biopsy approach for detection of tumor-derived alterations in the plasma in the setting of early detection and detection of minimal residual disease in stage II and III CRC patients.

We analyzed samples from 52 patients enrolled in the MEDOCC-PLCRC study, a prospective, observational study ongoing in the Netherlands to collect biospecimens from stage II and III CRC patients. Patients had a baseline blood draw at the time of diagnosis and were treatment naïve. Based on the current standard of care all stage II and III patients had a surgical resection during which tumor tissue was collected for genomic analyses. A post-resection liquid biopsy was collected between one and 12 weeks after surgery to allow time for the patient to heal and to define a window of therapeutic intervention where possible adjuvant therapy would be efficacious for patients in whom minimal residual disease was identified. Buffy coat was collected from the baseline liquid biopsy as source of white blood cells.

We first analyzed the baseline and post-resection liquid biopsies from the 52 patients in the study using the ultra-deep, targeted sequencing approach we previously developed (27). We identified mutations down to 0.05% mutant allele fraction when the mutation was present in least three distinct DNA molecules with three duplicate molecules having the identical base change (27). Each liquid biopsy was analyzed independently without knowledge of mutations identified in the other sample. In total, 152 mutations were identified in the 52 patients across both timepoints (Table 1 below). 128 mutations in 47 patients were identified at baseline while 76 mutations in 38 patients were identified post resection (Table 1 below). We hypothesized that a subset of the alterations in the plasma were tumor-derived, while a subset resulted from clonal hematopoiesis or germline changes. We next analyzed the matched white blood cells from the 52 patients using independent ultra-deep, targeted sequencing to identify mutations present in at least one distinct DNA molecule with at least three duplicate molecules having the identical mutation. Mutations in the baseline or post-resection plasma which were also identified in the matched white blood cells were removed from further analysis and considered hematopoietic or germline alterations. To limit the mutations in the plasma to tumor-derived mutations, we took a conservative approach and called mutations in the white blood cells to a much deeper level compared to the plasma analyses to remove any mutation that was present even at 0.01% mutant allele fraction in the white blood cells.

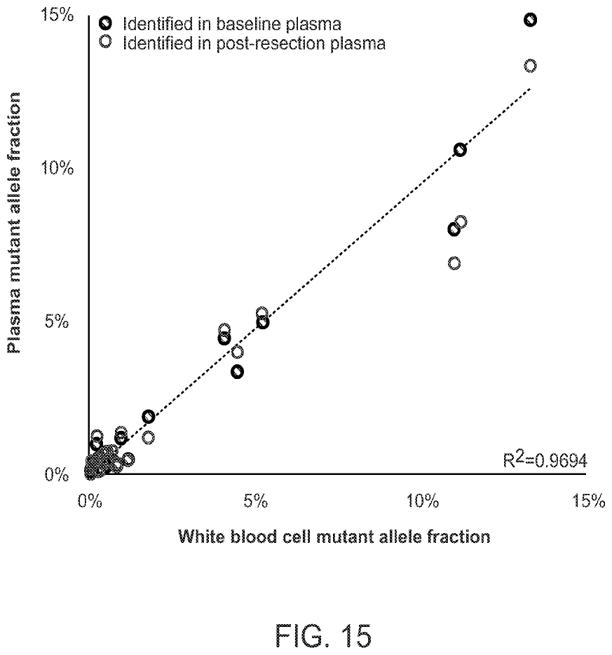

Mutations identified in white blood cells included variants in DNMT3A, a gene well-known to be altered in clonal hematopoiesis, as well as genes more commonly thought of as cancer drivers such as TP53, APC, and KRAS. We observed that the mutant allele fractions of alterations in the white blood cells and of the same alteration in baseline of post-resection plasma were highly correlated, R²=0.97 (FIG. 15). Hematopoietic mutations were also likely to be present in both the baseline and post-resection plasma samples at a similar mutant allele fraction.

After removing alterations from the plasma that were also identified in the matched white blood cells, 65 mutations in 28 patients remained in the baseline plasma and 13 mutations in eight patients remained in the post-resection plasma (Table 1 below). To further investigate the origin of these mutations, we analyzed matched tumor tissue using independent targeted sequencing to identify variants. We found that 50 of 65 mutations at baseline, 77%, in 23 patients, 82%, were concordant at high level (≥10%) in the matched tumor (Table 1 below). While tumor sequencing is not possible in the setting of early detection, confirmation of concordance between plasma and tumor alterations shows that the majority of mutations remaining after analysis using the white blood cell-guided approach are tumor derived and likely to be indicative of the presence of CRC.

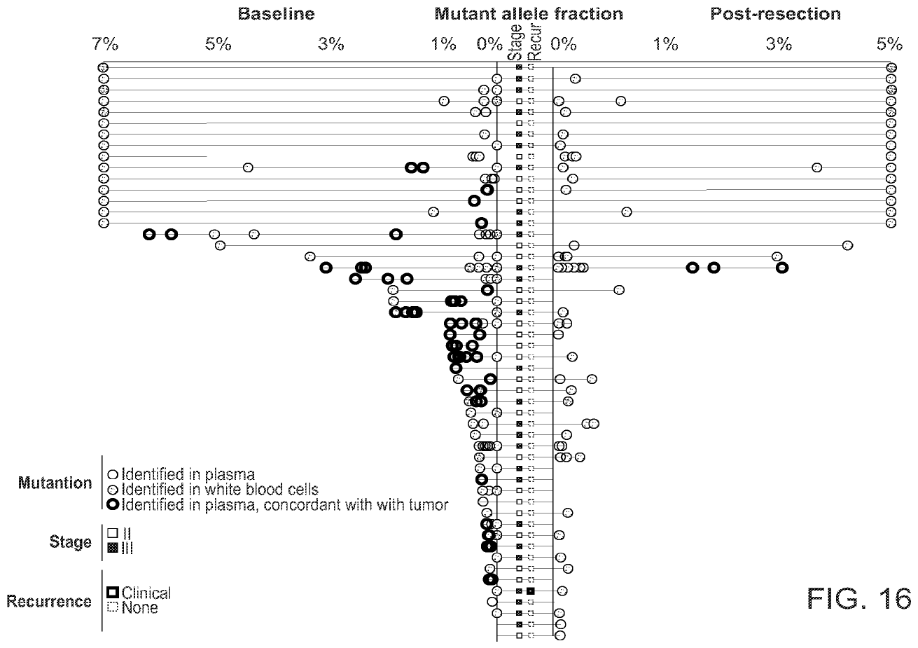

Using the white blood cell-guided approach for detection of tumor-derived mutations in plasma, we detected 28 of 52 patients at baseline, 54%, and 8 of 52 patients post resection, 15% (FIG. 16). Tumor-derived mutations ranged from 0.1% to 6.17% at baseline and 0.09% to 4.06% post resection. Mutations derived from clonal hematopoiesis were clustered at low mutant allele fractions, and germline changes above 25% were also evident through analyses of matched white blood cells (FIGS. 15 and 16). For patients with detected minimal residual disease post resection, n=8, we assessed whether a clinical recurrence had been documented; none of the eight patients with molecular recurrence had developed recurrent CRC using standard monitoring. While 20% of patients recur within 5 years, the follow up time for our cohort has reached only 2.5 years and it is possible that patients will recur as the timeline extends.

We have shown that early detection and detection of minimal residual disease in stage II and III colorectal cancer patients using a noninvasive, white blood cell-guided liquid biopsy approach to identify mutations as biomarkers is feasible and results in identification of tumor-derived mutations representative of disease. The removal of alterations in white blood cells is a necessary step to remove the background of germline and hematopoietic changes which confound plasma analyses. Our data suggest that high concordance of plasma mutations with those identified in the matched tumor can only be achieved through removal of white blood cell alterations identified in the white blood cells. Overall, white blood cell-guided liquid biopsy analyses have the potential to enhance the specificity of noninvasive detection of cancer in the settings of early detection and disease monitoring.

TABLE 1
Identification of mutations in baseline and post-resection plasma

| | | Cohort | | | |
|---|---|---|---|---|---|
| Analysis | | All | Recurrence | | |
| Mutations | Patients | 52 | 6 | | |
| identified in | Mutations | 152 | 18 | | |
| plasma samples | | | | | |
| | | Baseline plasma | | Post-resection plasma | |
| | | All | Recurrence | All | Recurrence |
| Mutations | Patients | 47 | 5 | 38 | 5 |
| identified in | Fraction detected | 90% | 83% | 73% | 83% |
| plasma | Mutations | 128 | 14 | 76 | 9 |
| timepoint | | | | | |
| Mutations | Patients | 28 | 3 | 8 | 0 |
| filtered to | Fraction detected | 54% | 50% | 15% | 0% |
| remove any | ≥1 mutation concordant with tumor | 23 | 3 | 1 | 0 |
| identified in | Fraction | 82% | 100% | 13% | NA |
| white blood cells | Mutations | 65 | 8 | 13 | 0 |
| | Concordant with tumor | 50 | 8 | 3 | 0 |
| | Fraction | 77% | 100% | 23% | 0% |

### REFERENCES FOR EXAMPLE 2

1. Jemal A, Bray F, Center M M, Ferlay J, Ward E, Forman D. Global cancer statistics. CA Cancer J Clin. 2011; 61(2):69-90. doi: 10.3322/caac.20107. PubMed PMID: 21296855.
2. Torre L A, Bray F, Siegel R L, Ferlay J, Lortet-Tieulent J, Jemal A. Global cancer statistics, 2012. CA Cancer J Clin. 2015; 65(2):87-108. doi: 10.3322/caac.21262. PubMed PMID: 25651787.
3. Organization WH. Guide to Cancer Early Diagnosis. Guide to Cancer Early Diagnosis. 2017.
4. Edwards B K, Ward E, Kohler B A, Eheman C, Zauber A G, Anderson R N, et al. Annual report to the nation on the status of cancer, 1975-2006, featuring colorectal cancer trends and impact of
5. interventions (risk factors, screening, and treatment) to reduce future rates. Cancer. 2010; 116(3):544-73. doi: 10.1002/cncr.24760. PubMed PMID: 19998273; PubMed Central PMCID: PMCPMC3619726.
6. Winawer S J. The multidisciplinary management of gastrointestinal cancer. Colorectal cancer screening. Best Pract Res Clin Gastroenterol. 2007; 21(6):1031-48. doi: 10.1016/j.bpg.2007.09.004. PubMed PMID: 18070702.
7. Ruibal Morell A. CEA serum levels in non-neoplastic disease. The International journal of biological markers. 1992; 7(3):160-6. PubMed PMID: 1431339.
8. Wanebo H J, Rao B, Pinsky C M, Hoffman R G, Stearns M, Schwartz M K, et al. Preoperative carcinoembryonic antigen level as a prognostic indicator in colorectal cancer. N Engl J Med. 1978; 299(9):448-51. doi: 10.1056/NEJM197808312990904. PubMed PMID: 683276.
9. Hol L, Wilschut J A, van Ballegooijen M, van Vuuren A J, van der Valk H, Reijerink J C, et al. Screening for colorectal cancer: random comparison of guaiac and immunochemical faecal occult blood testing at different cut-off levels. Br J Cancer. 2009; 100(7):1103-10. doi: 10.1038/sj.bjc.6604961. PubMed PMID: 19337257; PubMed Central PMCID: PMCPMC2670000.
10. Jin P, Kang Q, Wang X, Yang L, Yu Y, Li N, et al. Performance of a second-generation methylated SEPT9 test in detecting colorectal neoplasm. J Gastroenterol Hepatol. 2015; 30(5):830-3. doi: 10.1111/jgh.12855. PubMed PMID: 25471329.
11. Johnson D A, Barclay R L, Mergener K, Weiss G, Konig T, Beck J, et al. Plasma Septin9 versus fecal immunochemical testing for colorectal cancer screening: a prospective multicenter study. PLoS One. 2014;9(6):e98238. doi: 10.1371/journal.pone.0098238. PubMed PMID: 24901436; PubMed Central PMCID: PMCPMC4046970.
12. Edge, S. B. & Compton, C. C. The American Joint Committee on Cancer: the 7th edition of the AJCC cancer staging manual and the future of TNM. Ann Surg Oncol 17, 1471-1474, doi:10.1245/s10434-010-0985-4 (2010).
13. Quah, H. M. et al. Identification of patients with high-risk stage II colon cancer for adjuvant therapy. Diseases of the colon and rectum 51, 503-507, doi:10.1007/s10350-008-9246-z (2008).
14. Niedzwiecki, D. et al. Documenting the natural history of patients with resected stage II adenocarcinoma of the colon after random assignment to adjuvant treatment with edrecolomab or observation: results from CALGB 9581. J Clin Oncol 29, 3146-3152, doi:10.1200/JCO.2010.32.5357 (2011). 15. O'Connor, E. S. et al. Adjuvant chemotherapy for stage II colon cancer with poor prognostic features. J Clin Oncol 29, 3381-3388, doi:10.1200/JCO.2010.34.3426 (2011).
16. Figueredo, A., Charette, M. L., Maroun, J., Brouwers, M. C. & Zuraw, L. Adjuvant therapy for stage II colon cancer: a systematic review from the Cancer Care Ontario Program in evidence-based care's gastrointestinal cancer disease site group. J Clin Oncol 22, 3395-3407, doi:10.1200/JCO.2004.03.087 (2004).
17. Quasar Collaborative, G. et al. Adjuvant chemotherapy versus observation in patients with colorectal cancer: a randomised study. Lancet 370, 2020-2029, doi:10.1016/S0140-6736(07)61866-2 (2007).
18. Andre, T. et al. Adjuvant Fluorouracil, Leucovorin, and Oxaliplatin in Stage II to III Colon Cancer: Updated 10-Year Survival and Outcomes According to BRAF Mutation and Mismatch Repair Status of the MOSAIC Study. J Clin Oncol 33, 4176-4187, doi:10.1200/JCO.2015.63.4238 (2015).
19. Bettegowda C, Sausen M, Leary R J, Kinde I, Wang Y, Agrawal N, et al. Detection of circulating tumor DNA in early- and late-stage human malignancies. Sci Transl Med. 2014;6(224):224ra24. doi: 10.1126/scitranslmed.3007094. PubMed PMID: 24553385; PubMed Central PMCID: PMCPMC4017867.
20. Leary R J, Sausen M, Kinde I, Papadopoulos N, Carpten J D, Craig D, et al. Detection of chromosomal alterations in the circulation of cancer patients with whole-genome sequencing. Sci Transl Med. 2012;4(162):162ra54. doi: 10.1126/scitranslmed.3004742. PubMed PMID: 23197571; PubMed Central PMCID: PMC3641759.
21. Sausen M, Phallen J, Adleff V, Jones S, Leary R J, Barrett M T, et al. Clinical implications of genomic alterations in the tumour and circulation of pancreatic cancer patients. Nat Commun. 2015; 6:7686. doi: 10.1038/ncomms8686. PubMed PMID: 26154128.
22. Dawson S J, Tsui D W, Murtaza M, Biggs H, Rueda O M, Chin S F, et al. Analysis of circulating tumor DNA to monitor metastatic breast cancer. N Engl J Med. 2013; 368(13):1199-209. doi: 10.1056/NEJMoa1213261. PubMed PMID: 23484797.
23. Forshew T, Murtaza M, Parkinson C, Gale D, Tsui D W, Kaper F, et al. Non-invasive identification and monitoring of cancer mutations by targeted deep sequencing of plasma DNA. Sci Transl Med. 2012;4(136):136ra68. doi: 10.1126/scitranslmed.3003726. PubMed PMID: 22649089.
24. Murtaza M, Dawson S J, Tsui D W, Gale D, Forshew T, Piskorz A M, et al. Non-invasive analysis of acquired resistance to cancer therapy by sequencing of plasma DNA. Nature. 2013; 497(7447):108-12. doi: 10.1038/naturel2065. PubMed PMID: 23563269.
25. Newman A M, Bratman S V, To J, Wynne J F, Eclov N C, Modlin L A, et al. An ultrasensitive method for quantitating circulating tumor DNA with broad patient coverage. Nat Med. 2014; 20(5):548-54. doi: 10.1038/nm.3519. PubMed PMID: 24705333; PubMed Central PMCID: PMC4016134.
26. Newman A M, Lovejoy A F, Klass D M, Kurtz D M, Chabon J J, Scherer F, et al. Integrated digital error suppression for improved detection of circulating tumor DNA. Nat Biotechnol. 2016; 34(5):547-55. doi: 10.1038/nbt.3520. PubMed PMID: 27018799; PubMed Central PMCID: PMC4907374.
27. Phallen, J. et al. Direct detection of early-stage cancers using circulating tumor DNA. Sci Transl Med 9, doi:10.1126/scitranslmed.aan2415 (2017).

### Example 3. Matched Leukocyte DNA Guided Liquid Biopsy Approaches for Response Monitoring in the Context of Immunotherapy for Metastatic Lung Cancer Patients

We tested the clinical utility of our matched leukocyte DNA guided liquid biopsy approach in accurately determining ctDNA molecular responses as they relate to clinical response monitoring in the context of immunotherapy. Distinguishing which cfDNA mutations are truly tumor-derived versus originating from sub-clonal populations of non-cancerous hematopoietic cells, is imperative in the metastatic setting, as with age and exposures (including radiation and chemotherapy), blood cell sub-clones that contain somatic mutations can clonally expand¹⁻⁴. Degradation of these clonal hematopoiesis (CH) cells produces cfDNA containing mutations, which can often be in solid cancer driver genes such as TP53, and thus confound the interpretation of liquid biopsies⁵⁻⁹. To this end, we incorporated our matched leukocyte DNA guided liquid biopsy approach to interpret ctDNA dynamics and their predictive value in distinguishing responders from non-responders for immunotherapy¹⁰.

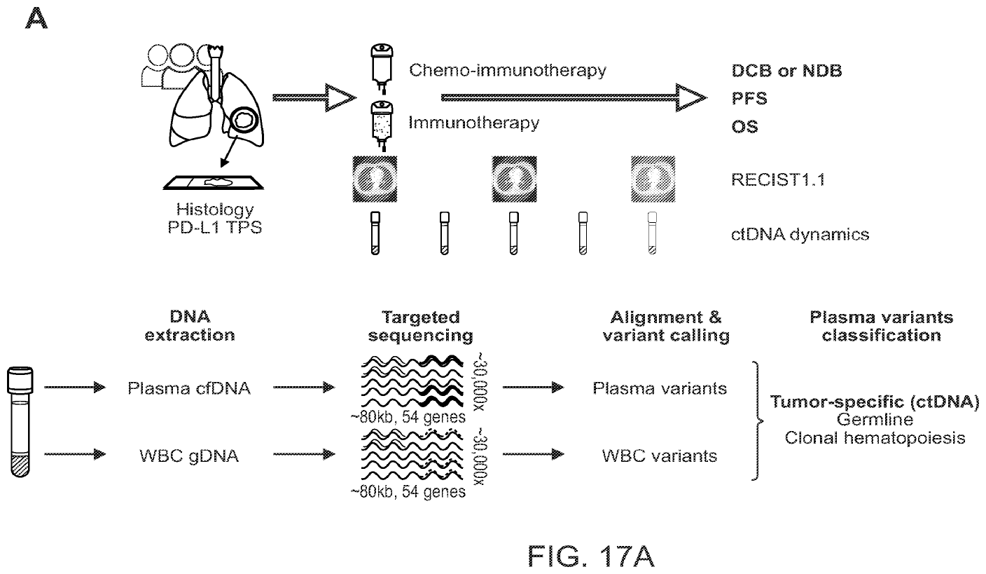

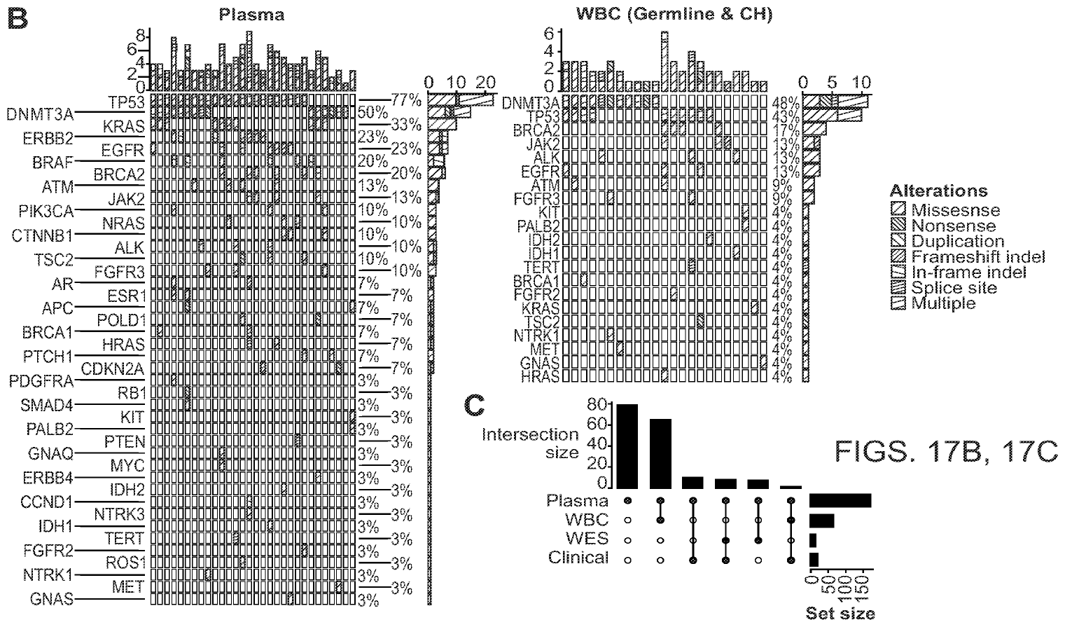

Cohort description. We designed a study to explore and model ctDNA dynamics during systemic treatment of non-small cell lung cancer (NSCLC) with immunotherapy-containing regimens to predict clinical outcomes (FIG. 17a)¹⁰. Using a prospective bio-specimen collection protocol of longitudinal plasma and whole blood specimens, we identified patients with advanced or metastatic non-small cell lung cancer treated with immunotherapy (IO) containing regimens. Plasma and whole blood specimens were collected at times where clinical venipuncture was indicated. When available, tumor tissue was collected for whole exome sequencing (WES). Clinical characteristics, including histopathology, PD-L1 status, and clinical tissue-based tumor mutational profiling (TMP), were collected in addition to response assessments including: radiographic response (RECIST1.1); progression-free and overall survival (PFS and OS); and durable or no durable clinical benefit (DCB or NDB) at 6 months.

A total of 31 patients were selected for inclusion in the study cohort that: (i) received at least one cycle of IO or chemo-IO treatment; (ii) had at least two plasma samples evaluable for cell-free DNA sequencing; (iii) had at least one whole blood sample evaluable for matched WBC sequencing; and (iv) had clinical follow-up through time of death or at least 6 months from time of treatment initiation. The cohort included patients with primarily smoking history (n=27 of 31), stage IV disease (n=28), adenocarcinoma histology (n=23), and positive PD-L1 tumor proportion score (TPS; n=20) across the treatment categories.

Classification of plasma variants. We performed deep targeted error correction sequencing of 142 plasma cell-free DNA (cfDNA) and 46 white blood cell genomic DNA (gDNA) specimens for the cohort of 31 patients. Plasma cfDNA sequencing was completed for baseline samples, prior to treatment, in 24 patients (range −2.6-0 weeks). Matched WBC sequencing was completed in 26 patients. For all reported samples, targeted capture libraries encompassing regions of 58 cancer-associated genes were subjected to ultrasensitive targeted sequencing followed by sequence alignment, error correction, and variant calling. A total of 160 variants in 38 genes were detected in plasma cfDNA and 66 variants in 21 genes in WBC gDNA (FIG. 1B). These plasma cfDNA variants were classified as WBC or ctDNA variants. In cases where matched plasma cfDNA and WBC gDNA were evaluable, plasma variants detected in WBC gDNA were classified as WBC variants and excluded as ctDNA variants. Using this classification system, 46% of plasma variants (n=74 of 160) were found only in the plasma (FIGS. 1B and 1C). Almost a third of the plasma cfDNA variants (32%, n=51 of 160) were deemed CH variants, confirming our previous findings regarding the diversity of the cfDNA pool and the importance of our paired plasma/matched leukocyte DNA sequencing approach to filter out clonal hematopoiesis alterations that may confound the assessment of molecular response. (FIGS. 17B and 17C).

As depicted in FIG. 17A we implemented biospecimen and clinical metadata collection protocols that allow for collection of serial blood and tissue samples from lung cancer patients treated with immunotherapy. Plasma and matched leukocyte DNA samples were deeply sequenced, clonal hematopoiesis variants were filtered out and ctDNA molecular responses were interpreted with respect to the clinical phenotypes of the patients. As shown in FIGS. 17B and 17C, we found cfDNA mutations from white blood cells in genes not canonically associated with clonal hematopoiesis, again highlighting the importance of deep sequencing of matched leukocyte DNA in order to determine whether cfDNA mutations are tumor or CH-derived.

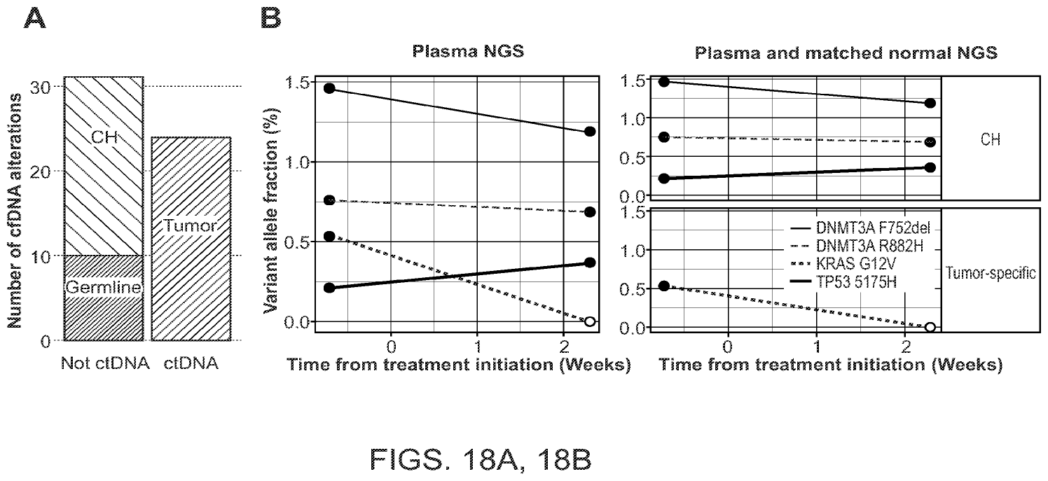

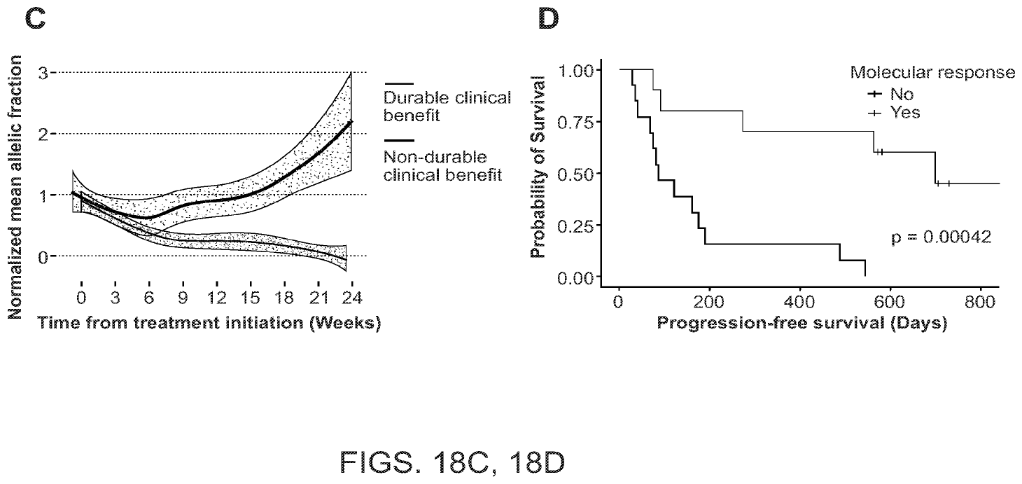

A representative example is shown in FIG. 18, for a patient responding to immunotherapy, with several variants detected by plasma next generation sequencing; appropriately classified into CH- and tumor-derived categories which allowed for assessment of molecular response 3 weeks after treatment initiation. Change in levels of tumor derived variants but not germline or clonal hematopoiesis derived variants were predictive of benefit to immune checkpoint blockade (FIGS. 2C and 2D). Overall, for our IO treated cohort, ctDNA dynamics accurately captured clinical response only after CH-derived mutations were filtered out.

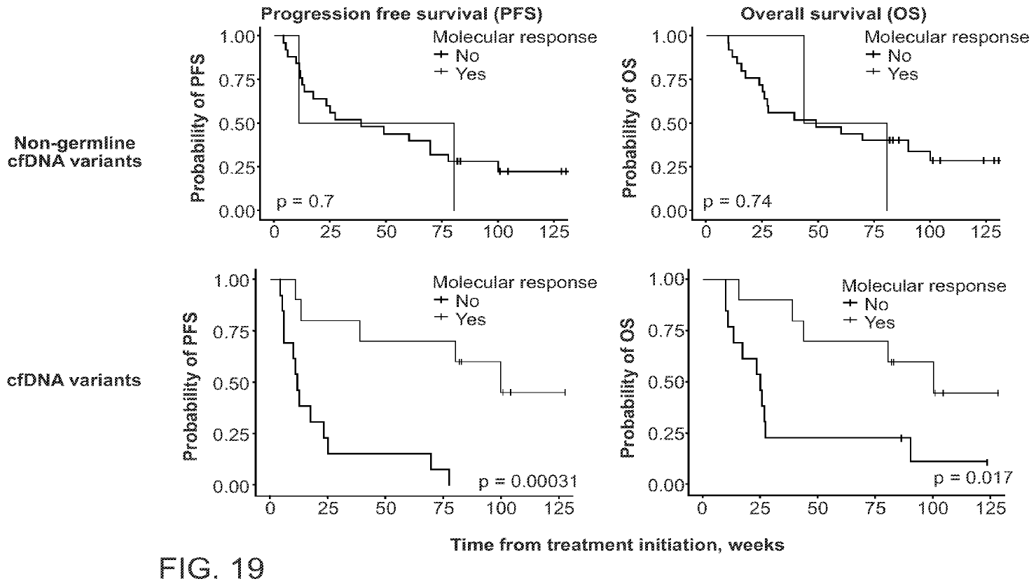

To further explore these findings and the importance of matched leukocyte DNA sequencing and analysis, we investigated the predictive and prognostic performance of ctDNA molecular responses with and without filtering out CH-derived mutations. As shown in FIG. 19, inclusion of CH-derived variants completely obliterated the clinical utility of ctDNA dynamics in predicting progression-free and overall survival.

As shown in FIG. 19, inclusion of CH-derived and germline mutations did not allow for accurate determination of ctDNA molecular response and ctDNA dynamics were not associated with progression-free and overall survil (top panel). In contrast, when matched leukocyte DNA deep sequencing was employed, variants were accurately classified in tumor-derived and CH-derived categories, which in turn allowed for distinction of molecular ctDNA responders from non-responders. Post filtering, ctDNA responders had a significantly longer progression-free and overall survival (bottom panel).

### Example 4: Matched Leukocyte DNA Guided Liquid Biopsy Approaches for Response Monitoring in the Context of Immunotherapy for Early Stage Esophageal Cancer Patients

The importance of our matched leukocyte DNA approach is also exemplified in the setting of disease monitoring for early stage esophageal cancer treated with combined immune checkpoint inhibition and chemo-radiotherapy. We examined the utility of serial liquid biopsies to monitor clonal dynamics and predict pathologic response in patients with esophageal/gastroesophageal junction (E/GEJ) cancer undergoing treatment with neoadjuvant immunotherapy and concurrent chemoradiation (CA209-906; NCT03044613). Using targeted error correction sequencing, we performed high-depth next generation sequencing on 79 serial plasma samples and matched leukocyte DNA from 16 patients with operable stage II/III E/GEJ cancer undergoing treatment with neoadjuvant nivolumab, followed by nivolumab plus chemoradiation and surgery as part of the CA209-906 trial. Liquid biopsies were evaluated pre-treatment, after each of two cycles of neoadjuvant nivolumab, and after concurrent nivolumab and chemoradiation immediately prior to surgery, for an average of 4 time points per patient.

For each plasma variant identified, we investigated whether these were also present in matched leukocyte DNA by deep next-generation sequencing. Variants identified in plasma and leukocyte DNA were considered germline or clonal hematopoiesis derived and further excluded from analyses. Eight of 16 patients had detectable circulating tumor-derived DNA (ctDNA) at any time point. Additionally, 13 CH-derived mutations were detected in plasma of eight patients. The number of CH-derived mutations was correlated with increasing patient age. Identification and removal of CH-derived mutations via comparison to matched leukocyte sequencing allowed for accurate assessment of kinetics of bona fide tumor-derived mutations in plasma.

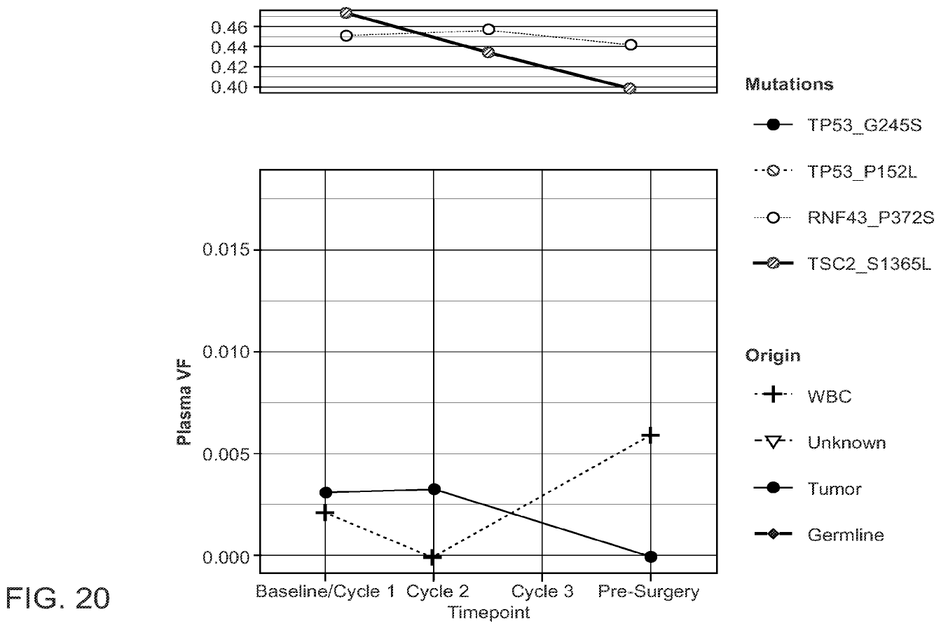

A representative example is shown in FIG. 20, where ctDNA dynamics captured by the tumor-derived TP53 G245S mutation were concordant with tumor regression noted at the time of resection (10% residual tumor) only when germline-derived and a TP53 CH-derived variant were excluded by matched leukocyte DNA sequencing analyses.

Post filtering, detectable ctDNA at the last pre-surgery time point was found in 3 patients and was associated with residual tumor>20% (50% vs 23% with or without detectable ctDNA respectively). ctDNA clearance, that is detectable ctDNA at one or more earlier time points that subsequently becomes undetectable before surgery, occurred in 5 patients and was associated with improved pathologic response (80% of patients with ctDNA clearance had residual tumor<=20% and no evidence of disease progression). Furthermore, of the three patients who did not have ctDNA clearance, two of them subsequently developed disease progression.

In summary, our new data summarized in parts I-III above provide additional evidence on the innovative aspects and clinical utility of our matched plasma-leukocyte DNA sequencing approach that is applicable to almost every stage of the management of patients with cancer, including diagnosis, the detection of residual disease and response monitoring and spans over multiple cancer types, stages of disease and therapeutic settings.

### REFERENCES FOR EXAMPLES 3 AND 4

1 Coombs, C. C. et al. Therapy-Related Clonal Hematopoiesis in Patients with Non-hematologic Cancers Is Common and Associated with Adverse Clinical Outcomes. Cell stem cell 21, 374-382 e374, doi:10.1016/j.stem.2017.07.010 (2017).
2 Acuna-Hidalgo, R. et al. Ultra-sensitive Sequencing Identifies High Prevalence of Clonal Hematopoiesis-Associated Mutations throughout Adult Life. American journal of human genetics 101, 50-64, doi:10.1016/j.ajhg.2017.05.013 (2017).
3 Jaiswal, S. et al. Age-related clonal hematopoiesis associated with adverse outcomes. N Engl J Med 371, 2488-2498, doi:10.1056/NEJMoa1408617 (2014).
4 Zink, F. et al. Clonal hematopoiesis, with and without candidate driver mutations, is common in the elderly. Blood 130, 742-752, doi:10.1182/blood-2017-02-769869 (2017).
5 Leal, A. et al. White blood cell and cell-free DNA analyses for detection of residual disease in gastric cancer. Nature communications 11, 525, doi:10.1038/s41467-020-14310-3 (2020).
6 Abbosh, C., Swanton, C. & Birkbak, N. J. Clonal haematopoiesis: a source of biological noise in cell-free DNA analyses. Annals of oncology: official journal of the European Society for Medical Oncology 30, 358-359, doi:10.1093/annonc/mdy552 (2019).
7 Hu, Y. et al. False-Positive Plasma Genotyping Due to Clonal Hematopoiesis. Clinical cancer research: an official journal of the American Association for Cancer Research 24, 4437-4443, doi:10.1158/1078-0432.CCR-18-0143 (2018).
8 Liu, J. et al. Biological background of the genomic variations of cf-DNA in healthy individuals. Annals of oncology: official journal of the European Society for Medical Oncology 30, 464-470, doi:10.1093/annonc/mdy513 (2019).
9 Razavi, P. et al. High-intensity sequencing reveals the sources of plasma circulating cell-free DNA variants. Nature medicine 25, 1928-1937, doi:10.1038/s41591-019-0652-7 (2019).
10 Murray, J. et al. Comprehensive modeling of longitudinal circulating tumor DNA dynamics to predict clinical response to first-line immunotherapy and chemoimmunotherapy in advanced non-small cell lung cancer. Journal of Clinical Oncology 38, 9525-9525 (2020).

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. Genbank and NCBI submissions indicated by accession number cited herein are hereby incorporated by reference. All other published references, documents, manuscripts and scientific literature cited herein are hereby incorporated by reference.

While this invention has been particularly shown and described with references to preferred embodiments thereof, it will be understood by those skilled in the art that various changes in form and details may be made therein without departing from the scope of the invention encompassed by the appended claims.

1. A method of detecting gastric, colorectal, lung and/or esophageal tumor specific mutations in a subject's circulating tumor DNA, comprising:
preparing sequencing libraries of genomic DNA comprising cell free DNA (cfDNA) and cellular DNA obtained from a sample of the subject's whole blood;
identifying sequence variations in the cfDNA and cellular DNA as compared to a reference genomic sequence;
comparing the sequence variations of cfDNA and cellular DNA; thereby, identifying gastric tumor specific mutations.

2. (canceled)

3. The method of claim 1, wherein the cfDNA is obtained from a plasma component of the subject's whole blood.

4. The method of claim 3, wherein the cellular DNA is obtained from white blood cells of the subject's whole blood.

5. The method of claim 1, wherein sequence specific mutations detected in both cfDNA and white blood cell DNA are excluded as tumor specific mutations.

6. The method of claim 1, wherein sequence specific mutations detected exclusively in cfDNA are identified as tumor specific mutations.

7. The method of claim 1, wherein the tumor specific mutations are indicative of cancer.

8. The method of claim 1, wherein the tumor specific mutations are indicative of gastric cancer.

9. The method of claim 1, wherein the tumor specific mutations are indicative of colorectal cancer.

10. The method of claim 1, wherein the tumor specific mutations are indicative of lung cancer.

11. The method of claim 1, wherein the tumor specific mutations are indicative of esophageal cancer.

12. (canceled)

13. The method of claim 6, wherein cfDNA tumor specific mutations are detected in one or more genes comprising: PIK3CA, ATM, KRAS, PIK3CA, PTEN, BRAF, ERBB4, CDH1, KIT, MYC, SMAD4, ERBB2, PTEN, EGFR, KIT, CDK4, JAK2, PIK3R1, AR, MYC, STK11, TP53, HRAS, FBXW7, ABL1, ALK, CTNNB1, APC, PIK3R1, JAK2 or combinations thereof.

14. The method of claim 13, wherein cfDNA tumor specific mutations are detected in one or more genes comprising: TP53, MYC, PIK3CA, KRAS, HRAS, ALK, ATM, KIT, CDII or combinations thereof.

15. A method of predicting clinical outcome of a subject suffering from gastric cancer, colorectal, lung and/or esophageal comprising:
obtaining whole blood sample from the subject;
separating plasma and cellular components to obtain cell free DNA and cellular DNA; preparing sequencing libraries of the cell free DNA (cfTDNA) and the cellular DNA; identifying sequence variations in the cfDNA and cellular DNA as compared to a reference genomic sequence; comparing the sequence variations of cfDNA and cellular DNA to identify sequence mutations cfDNA; thereby,
predicting clinical outcome of a subject suffering from gastric cancer.

16. (canceled)

17. The method of claim 15, wherein the cellular DNA is obtained from white blood cells.

18. The method of claim 15, wherein sequence specific mutations detected in both cfDNA and white blood cell DNA are excluded as tumor specific mutations.

19. The method of claim 15, wherein sequence specific mutations detected exclusively in cfDNA are identified as tumor specific mutations.

20. The method of claim 19, wherein detection of tumor specific mutations in cfDNA is indicative of a high risk of recurrence of the cancer in the subject.

21-26. (canceled)

27. The method of claim 26, wherein cfDNA tumor specific mutations are detected in one or more genes comprising: PIK3CA, ATM, KRAS, PIK3CA, PTEN, BRAF, ERBB4, CDH1, KIT, MYC, SMAD4, ERBB32, PTEN, EGFR, KIT, CDK4, JAK2, PIK3R1, AR, MYC, STK11, TP53, HRAS, FBXW7, ABL1, ALK, CTNNB1, APC, PIK3R1, JAK2 or combinations thereof.

28. The method of claim 27, wherein cfDNA tumor specific mutations are detected in one or more genes comprising: TP53, MYC, PIK3CA, KRAS, HRAS, ALK, ATM, KIT, CDH1 or combinations thereof.

29.-39. (canceled)
